SEQUENCE LISTING (57 sequences)

SEQ ID NO: 1 - DNA, 111 bp, Homo sapiens
ttgggggccccttccccacactatctcaatgcaaatatctgtctgaaacggtccctggctaaactccacccatgggttggccagccttgccttgaccaatagccttgacaa

SEQ ID NO: 2 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1368 aa, Artificial; identical to SEQ ID NO: 2
  Synthetic Nickase (D10A)

SEQ ID NO: 4 - protein, 198 aa, Homo sapiens [sequence omitted]

SEQ ID NO: 5 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 6 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 7 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 8 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 9 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 10 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 11 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 12 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 13 - protein, 191 aa, Homo sapiens
MEAKAAPKPAASGACSVSAEETEKWMEEAMHMAKEALENTEVPVGCLMVYNNEVVGKGRNEVNQTKNATRHAEMVAIDQVLDWCRQSGKSPSEVFEHTVLYVTVEPCIMCAAALRLMKIPLVVYGCQNERFGGCGSVLNIASADLPNTGRPFQCIPGYRAEEAVEMLKTFYKQENPNAPKSKVRKKECQKS

SEQ ID NO: 14 - protein, 502 aa, Homo sapiens
MWTADEIAQLCYEHYGIRLPKKGKPEPNHEWTLLAAVVKIQSPADKACDTPDKPVQVTKEVVSMGTGTKCIGQSKMRKNGDILNDSHAEVIARRSFQRYLLHQLQLAATLKEDSIFVPGTQKGVWKLRRDLIFVFFSSHTPCGDASIIPMLEFEDQPCCPVFRNWAHNSSVEASSNLEAPGNERKCEDPDSPVTKKMRLEPGTAAREVTNGAAHHQSFGKQKSGPISPGIHSCDLTVEGLATVTRIAPGSAKVIDVYRTGAKCVPGEAGDSGKPGAAFHQVGLLRVKPGRGDRTRSMSCSDKMARWNVLGCQGALLMHLLEEPIYLSAVVIGKCPYSQEAMQRALIGRCQNVSALPKGFGVQELKILQSDLLFEQSRSAVQAKRADSPGRLVPCGAAISWSAVPEQPLDVTANGFPQGTTKKTIGSLQARSQISKVELFRSFQKLLSRIARDKWPHSLRVQKLDTYQEYKEAASSYQEAWSTLRKQVFGSWIRNPPDYHQFK

SEQ ID NO: 15 - DNA, 23 bp, Homo sapiens
atatttgcattgagatagtgtgg

SEQ ID NO: 16 - DNA, 23 bp, Homo sapiens
gtggggaaggggcccccaagagg

SEQ ID NO: 17 - DNA, 23 bp, Homo sapiens
cttgaccaatagccttgacaagg

SEQ ID NO: 18 - DNA, 22 bp, Homo sapiens
ttgaccaatagccttgacaagg

SEQ ID NO: 19 - DNA, 22 bp, Homo sapiens
ccttccccacactatctcaatg

SEQ ID NO: 20 - DNA, 23 bp, Homo sapiens
gccccttccccacactatctcaa

SEQ ID NO: 21 - DNA, 30 bp, Artificial
  Synthetic primer
aaaaacggctgacaaaagaagtcctggtat

SEQ ID NO: 22 - DNA, 30 bp, Artificial
  Synthetic primer
ataacctcagacgttccagaagcgagtgtg

SEQ ID NO: 23 - DNA, 29 bp, Artificial
  Synthetic primer
gttttaaaacaacaaaaatgagggaaaga

SEQ ID NO: 24 - DNA, 27 bp, Artificial
  Synthetic primer
gttgctttataggatttttcactacac

SEQ ID NO: 25 - DNA, 20 bp, Artificial
  Synthetic primer
cccttccgagaggatttagg

SEQ ID NO: 26 - DNA, 20 bp, Artificial
  Synthetic primer
agtcgggatctgaacaatgg

SEQ ID NO: 27 - DNA, 21 bp, Artificial
  Synthetic primer
cggtcaacttcaagctcctaa

SEQ ID NO: 28 - DNA, 20 bp, Artificial
  Synthetic primer
acagaagccaggaacttgtc

SEQ ID NO: 29 - DNA, 22 bp, Artificial
  Synthetic primer
gcaaggtgaacgtggatgaagt

SEQ ID NO: 30 - DNA, 23 bp, Artificial
  Synthetic primer
taacagcatcaggagtggacaga

SEQ ID NO: 31 - DNA, 19 bp, Artificial
  Synthetic primer
cctgtcctctgcctctgcc

SEQ ID NO: 32 - DNA, 19 bp, Artificial
  Synthetic primer
ggattgccaaaacggtcac

SEQ ID NO: 33 - protein, 1368 aa, Artificial
  Synthetic Nickase (D840A)
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 34 - DNA, 6 bp, Homo sapiens
cagata

SEQ ID NO: 35 - DNA, 6 bp, Homo sapiens
  misc_feature (6)..(6): N is G or A
cagatn

SEQ ID NO: 36 - DNA, 4 bp, Homo sapiens
cttc

SEQ ID NO: 37 - DNA, 4 bp, Homo sapiens
  misc_feature (3)..(3): N is T or C
ctnc

SEQ ID NO: 38 - DNA, 6 bp, Homo sapiens
tgacca

SEQ ID NO: 39 - DNA, 9 bp, Homo sapiens
  misc_feature (3)..(3): N is G or A
  misc_feature (6)..(6): N is G or A
tgnccnata

SEQ ID NO: 40 - DNA, 6 bp, Homo sapiens
  misc_feature (4)..(4): N is T or C
  misc_feature (5)..(5): N is T or C
tganna

SEQ ID NO: 41 - DNA, 10 bp, Homo sapiens
ccccttcccc

SEQ ID NO: 42 - DNA, 10 bp, Homo sapiens
  misc_feature (1)..(1): N is C or T
  misc_feature (2)..(2): N is C or T
  misc_feature (3)..(3): N is C or T
  misc_feature (4)..(4): N is C or T
  misc_feature (7)..(7): N is C or T
  misc_feature (8)..(8): N is C or T
  misc_feature (9)..(9): N is C or T
  misc_feature (10)..(10): N is C or T
nnnnttnnnn

SEQ ID NO: 43 - DNA, 10 bp, Homo sapiens
  misc_feature (3)..(3): N is C or T
  misc_feature (4)..(4): N is C or T
  misc_feature (7)..(7): N is C or T
  misc_feature (8)..(8): N is C or T
  misc_feature (9)..(9): N is C or T
  misc_feature (10)..(10): N is C or T
ccnnttnnnn

SEQ ID NO: 44 - DNA, 10 bp, Homo sapiens
  misc_feature (3)..(3): N is C or T
  misc_feature (7)..(7): N is C or T
  misc_feature (8)..(8): N is C or T
  misc_feature (9)..(9): N is C or T
  misc_feature (10)..(10): N is C or T
ccncttnnnn

SEQ ID NO: 45 - DNA, 6 bp, Homo sapiens
tgacca

SEQ ID NO: 46 - DNA, 10 bp, Homo sapiens
  misc_feature (7)..(7): N is C or T
  misc_feature (8)..(8): N is C or T
  misc_feature (9)..(9): N is C or T
  misc_feature (10)..(10): N is C or T
ccccttnnnn

SEQ ID NO: 47 - DNA, 10 bp, Homo sapiens
  MISC (4)..(4): N is C or T
  misc_feature (4)..(4): n is a, c, g, or t
cccnttcccc

SEQ ID NO: 48 - RNA, 20 nt, Artificial
  Synthetic gRNA
auauuugcauugagauagug

SEQ ID NO: 49 - RNA, 20 nt, Artificial
  Synthetic gRNA
guggggaaggggcccccaag

SEQ ID NO: 50 - RNA, 20 nt, Artificial
  Synthetic gRNA
cuugaccaauagccuugaca

SEQ ID NO: 51 - RNA, 22 nt, Artificial
  Synthetic gRNA
uugaccaauagccuugacaagg

SEQ ID NO: 52 - RNA, 20 nt, Artificial
  Synthetic gRNA
ccuuccccacacuaucucaa

SEQ ID NO: 53 - RNA, 20 nt, Artificial
  Synthetic gRNA
gccccuuccccacacuaucu

SEQ ID NO: 54 - DNA, 26 bp, Artificial Sequence
  Synthetic primer
acggataagtagatattgaggtaagc

SEQ ID NO: 55 - DNA, 21 bp, Artificial Sequence
  Synthetic primer
gtctctttcagttagcagtgg

SEQ ID NO: 56 - DNA, 20 bp, Artificial Sequence
  Synthetic primer
actcatgggagctgctggtt

SEQ ID NO: 57 - DNA, 21 bp, Artificial Sequence
  Synthetic primer
gctgtcatctcttgtgggctg

(19) United States
(12) Patent Application Publication
(10) Pub. No.: US 2023/0279438 A1
(43) Pub. Date: Sep. 7, 2023

(54) BASE EDITING APPROACHES FOR THE TREATMENT OF BETAHEMOGLOBINOPATHIES

(71) Applicants: INSERM (Institut National de la Santé et la Recherche Médicale), Paris (FR); Université Paris Cité, Paris (FR); Assistance Publique-Hôpitaux de Paris, Paris (FR); Fondation Imagine, Paris (FR)

(72) Inventors: Annarita MICCIO, Paris (FR); Panagiotis ANTONIOU, Paris (FR); Marina CAVAZZANA, Paris (FR)

(21) Appl. No.: 17/998,603

(22) PCT Filed: May 12, 2021
(86) PCT No.: PCT/EP2021/062633

§ 371 Date: Nov. 11, 2022

(30) Foreign Application Priority Data
May 13, 2020 (EP) 20305488.7

(51) Int. Cl. C12N 15/90; C12N 9/78; C12N 9/22; C12N 15/11

(52) U.S. Cl. CPC C12N 15/90; C12N 9/22; C12N 9/78; C12N 15/11; C12Y 305/04005; C12N 2310/20; C12N 2320/34

(57) ABSTRACT

The clinical history of β-hemoglobinopathies shows that the severity is mitigated by the synthesis of the fetal γ-globin in adulthood, typically associated with genetic variants the HBB cluster known as hereditary persistence of fetal hemoglobin (HPFH) mutations. The inventors identified that most of the known HPFH mutations in the γ-globin promoters (C>T, G>A, T>C or A>G) can be recapitulated using CBE- and ABE-mediatedbase-editing approaches. In particular, the inventors designed gRNAs that, when combined with CBEs or ABEs, generate HPFH mutations, and either disrupt binding sites for transcriptional repressors (-200 and -115 sites) or generate de novo DNA motifs recognized by transcriptional activators (e.g., -198 T>C, the -175 T>C and -113 A>G). It is noteworthy that a subset of the gRNAs targeting the -200 and the 115 regions are predicted to generate simultaneously HPFH mutations and also to make base changes other than HPFH mutations in or around the LRF and BCL11A binding sites, which might further reduce LRF and BCL11A occupancy. Accordingly, the present invention relates to base editing approaches for the treatment of β-hemoglobinopathies.

### FIELD OF THE INVENTION

The present invention is in the field of medicine, in particular haematology.

### BACKGROUND OF THE INVENTION

β-hemoglobinopathies, β-thalassemia and sickle cell disease (SCD), are monogenic diseases caused by mutations in the β-globin locus, affecting the synthesis or the structure of the adult hemoglobin (Hb). β-thalassemia is caused by mutations in the β-globin gene (HBB) locus that reduce (β⁺) or abolish (β⁰) the production of β-globin chains included in the adult hemoglobin (HbA) tetramer, leading to the precipitation of uncoupled α-globin chains, erythroid cell death and severe anemia ⁽¹⁾. In SCD, an A>T mutation in the HBB gene causes the substitution of valine for glutamic acid at position 6 of the β-globin chain (β^{S}), which is responsible for deoxygenation-induced polymerization of sickle hemoglobin (HbS). This primary event drives red blood cell (RBC) sickling, hemolysis, vaso-occlusive crises, multiorgan damage, often associated with severely reduced life expectancy⁽²⁾.

The only definitive cure for β-hemoglobinopathies is transplantation of allogeneic hematopoietic stem cells (HSCs) from an HLA-compatible donor, an option available to <30% of the patients⁽³⁾. Gene therapy approaches based on the transplantation of autologous, genetically modified HSCs have been investigated as a treatment option for patients lacking a compatible donor⁽⁴⁾. Genome editing technology has been exploited to develop therapeutic approaches for β-hemoglobinopathies, based on direct gene correction. These approaches use designer nucleases, such as the CRISPR/Cas9 system that induces DNA double-strand breaks (DSBs) via a single guide RNA (gRNA) complementary to a specific genomic target⁽⁴⁾.

The clinical history of β-hemoglobinopathies shows that the severity of both β-thalassemia and SCD is mitigated by the synthesis of the fetal γ-globin in adulthood, typically associated with genetic variants (deletions or point mutations) in the HBB cluster known as hereditary persistence of fetal hemoglobin (HPFH) mutations⁽⁵⁾. Fetal hemoglobin (HbF) compensates for the HbA deficiency in β-thalassemia, and γ-globin exerts a potent antisickling effect in SCD by replacing the mutant sickle β-chain⁽⁴⁾. In particular, mutations in the two identical promoters of the γ-globin genes (HBG1 and HBG2) either generate de novo DNA motifs recognized by transcriptional activators (TAL1, KLF1 and GATA1)^{(6,7,8)} or disrupt binding sites for transcriptional repressors (LRF and BCL11A)⁽⁹⁾. Several genome-editing strategies have been developed with the goal of reactivating the expression of fetal γ-globin as a potential therapy for both β-thalassemia and SCD, based on the disruption of cis-regulatory elements via the generation of deletions or insertions, mimicking HPFH mutations in patient hematopoietic stem/progenitor cells (HSPCs) and reactivate HbF expression in their erythroid progeny⁽¹⁰⁾. CRISPR/Cas9 disruption of LRF and BCL11A repressor binding sites in the γ-globin promoters efficiently reactivates HbF expression and ameliorates the phenotype of SCD RBCs⁽¹⁾. However, a large fraction of deletions that disrupt repressor binding sites are caused by MMEJ, which might not be effective in long-term repopulating, quiescent HSC fraction of the HSPC population, which is mostly composed of dividing progenitors^{(11,12)}.

It is noteworthy that HSCs are highly sensitive to DNA DSBs⁽¹³⁾ - especially in cases of multiple on-targets or concomitant on-target and off-target events. Even when highly specific gRNAs are used, Cas9/gRNA treatment of human HSPCs induces a DNA damage response that can lead to apoptosis⁽¹⁴⁾. CRISPR/Cas9 can cause P53-dependent cell toxicity and cell cycle arrest, resulting in the negative selection of cells with a functional P53 pathway⁽¹⁵⁾. Furthermore, the generation of several on-target DSBs, simultaneous on-target and off-target DSBs, or even a single on-target DSB is associated with a risk of deletion, inversion and translocation⁽¹⁶⁾. Hence, the development of novel, efficacious and safe therapeutic strategies for β-hemoglobinopathies based on precise base editing rather than on DSB-induced DNA repair has been preferential.

It has recently been shown that CRISPR-system-based cytosine and adenine base-editing enzymes (CBEs and ABEs) can make pinpoint changes in DNA with little or no DSB generation⁽¹⁷⁾. The basic components of base-editing enzymes are a catalytically disabled Cas9 nuclease and a deaminase; these eventually produce a C-G to T-A or A-T to G-C conversion (for CBEs and ABEs, respectively)⁽¹⁸⁾. Base-editing approaches allow precise DNA repair virtually in the absence of DSBs, and thus eliminate the risks of DSB-induced apoptosis, translocations and insertions or deletions of large portions of DNA. Furthermore, BEs have a lower level of off-target activity than Cas9 nuclease⁽¹⁷⁾. Importantly, base editing occurs in quiescent cells - suggesting that bona ƒide HSCs could be genetically modified using this novel technology⁽¹⁹⁾, and results in homogeneous, predictable base changes as compared to the heterogeneous and unpredictable mutagenesis induced by NHEJ.

### SUMMARY OF THE INVENTION

The present invention is defined by the claims. In particular, the present invention relates to base editing approaches for the treatment of β-hemoglobinopathies.

### DETAILED DESCRIPTION OF THE INVENTION

The inventors identified that most of the known HPFH mutations in the γ-globin promoters (C>T, G>A, T>C or A>G) can be recapitulated using CBE- and ABE-mediated base-editing approaches (FIG. 1). Compared with a CRISPR/Cas9-nuclease-based strategy, a base-editing approach might allow the simultaneous targeting of multiple regions in the γ-globin promoters, e.g., the generation of HPFH mutations that disrupt both -200 and -115 binding sites. This might have an additive effect on HbF reactivation, given the independent role of LRF and BCL11A in γ-globin repression⁽²⁰⁾. In contrast, a Cas9-nuclease-based strategy targeting two different regions of the γ-globin promoters (e.g., -200 and -115) would probably trigger the deletion of the intervening sequence, which could be detrimental for promoter activity⁽⁹⁾. Furthermore, base editing can be designed to generate multiple de novo binding sites for transcriptional activators (e.g., -198 T>C, the -175 T>C and -113 A>G)⁽²¹⁾. Importantly, this strategy would avoid the potential Cas9-nuclease-mediated deletion of the region between HBG1 and HBG2 promoters resulting from the simultaneous cleavage at the two γ-globin promoters that would lead to the loss of HBG2 expression⁽²²⁾. The inventors previously showed that this type of deletion occurs at a frequency of 10-15% in Cas9 nuclease-edited HUDEP-2⁽¹¹⁾. Importantly, the base-editing approach does not rely on MMEJ pathway and will avoid the potential DSB-induced toxicity. Thus, the inventors designed gRNAs that, when combined with CBEs or ABEs, generate HPFH mutations, and either disrupt binding sites for transcriptional repressors (-200 and -115 sites) or generate de novo DNA motifs recognized by transcriptional activators (e.g., -198 T>C, the -175 T>C and -113 A>G) (FIG. 1). It is noteworthy that a subset of the gRNAs targeting the -200 and the 115 regions are predicted to generate simultaneously HPFH mutations and also to make base changes other than HPFH mutations in or around the LRF and BCL11A binding sites, which might further reduce LRF and BCL11A occupancy⁽²³⁾.

### Definitions

As used herein, the term “β-hemoglobinopathy” has its general meaning in the art and refers to any defect in the structure or function of any hemoglobin of an individual, and includes defects in the primary, secondary, tertiary or quaternary structure of hemoglobin caused by any mutation, such as deletion mutations or substitution mutations in the coding regions of the HBB gene, or mutations in, or deletions of, the promoters or enhancers of such gene that cause a reduction in the amount of hemoglobin produced as compared to a normal or standard condition.

As used herein, the term “sickle cell disease” has its general meaning in the art and refers to a group of autosomal recessive genetic blood disorders, which results from mutations in a globin gene and which is characterized by red blood cells that assume an abnormal, rigid, sickle shape. They are defined by the presence of βS-globin gene coding for a β-globin chain variant in which glutamic acid is substituted by valine at amino acid position 6 of the peptide: incorporation of the βS-globin in the Hb tetramers (HbS, sickle Hb) leads to Hb polymerization and to a clinical phenotype. The term includes sickle cell anemia (HbSS), sickle-hemoglobin C disease (HbSC), sickle beta-plus- thalassaemia (HbS/β+), or sickle beta-zerothalassaemia (HbS/β0).

As used herein, the term “β-thalassemia” refers to a hemoglobinopathy that results from an altered ratio of α-globin to β-like globin polypeptide chains resulting in the underproduction of normal hemoglobin tetrameric proteins and the precipitation of free, unpaired α-globin chains.

As used herein, the term “hematopoietic stem cell” or “HSC” refers to blood cells that have the capacity to self-renew and to differentiate into precursors of blood cells. These precursor cells are immature blood cells that cannot self-renew and must differentiate into mature blood cells. Hematopoietic stem progenitor cells display a number of phenotypes, such as Lin-CD34+CD38-CD90+CD45RA-, Lin-CD34+CD38-CD90-CD45RA-, Lin- CD34+CD38+IL-3aloCD45RA-, and Lin-CD34+CD38+CD10+(Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-renew and maintain continuous production of hematopoietic stem cells that give rise to all mature blood cells throughout life. In some embodiments, the hematopoietic progenitor cells or hematopoietic stem cells are isolated form peripheral blood cells.

As used herein, the term “peripheral blood cells” refer to the cellular components of blood, including red blood cells, white blood cells, and platelets, which are found within the circulating pool of blood. In some embodiments, the eukaryotic cell is a bone marrow derived stem cell.

As used herein the term “bone marrow-derived stem cells” refers to stem cells found in the bone marrow. Stem cells may reside in the bone marrow, either as an adherent stromal cell type that possess pluripotent capabilities, or as cells that express CD34 or CD45 cell-surface protein, which identifies hematopoietic stem cells able to differentiate into blood cells.

As used herein, the term “mobilization” or “stem cell mobilization” refers to a process involving the recruitment of stem cells from their tissue or organ of residence to peripheral blood following treatment with a mobilization agent. This process mimics the enhancement of the physiological release of stem cells from tissues or organs in response to stress signals during injury and inflammation. The mechanism of the mobilization process depends on the type of mobilization agent administered. Some mobilization agents act as agonists or antagonists that prevent the attachment of stem cells to cells or tissues of their microenvironment. Other mobilization agents induce the release of proteases that cleave the adhesion molecules or support structures between stem cells and their sites of attachment.

As used herein, the term “mobilization agent” refers to a wide range of molecules that act to enhance the mobilization of stem cells from their tissue or organ of residence, e.g., bone marrow (e.g., CD34+ stem cells) and spleen (e.g., Hox11+ stem cells), into peripheral blood. Mobilization agents include chemotherapeutic drugs, e.g., cyclophosphamide and cisplatin; cytokines, and chemokines, e.g., granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1); agonists of the chemokine (C— C motif) receptor 1 (CCR1), such as chemokine (C—C motif) ligand 3 (CCL3, also known as macrophage inflammatory protein-1α (Mip-1α)); agonists of the chemokine (C—X—C motif) receptor 1 (CXCR1) and 2 (CXCR2), such as chemokine (C—X—C motif) ligand 2 (CXCL2) (also known as growth-related oncogene protein-β (Gro-β)), and CXCL8 (also known as interleukin-8 (IL-8)); agonists of CXCR4, such as CTCE-02142, and Met-SDF-1,; Very Late Antigen (VLA)-4 inhibitors; antagonists of CXCR4, such as TG-0054, plerixafor (also known as AMD3100), and AMD3465, or any combination of the previous agents. A mobilization agent increases the number of stem cells in peripheral blood, thus allowing for a more accessible source of stem cells for use in transplantation, organ repair or regeneration, or treatment of disease.

As used herein, the term “isolated cell” refers to a cell that has been removed from an organism in which it was originally found, or a descendant of such a cell. Optionally the eukaryotic cell has been cultured in vitro, e.g., in the presence of other cells. Optionally the eukaryotic cell is later introduced into a second organism or reintroduced into the organism from which it (or the cell from which it is descended) was isolated. As used herein, the term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

As used herein, the term “gamma globin” or “y-globin” has its general meaning in the art and refers to protein that is encoded in human by the HBG1 and HBG2 genes. The HBG1 and HBG2 genes are normally expressed in the fetal liver, spleen and bone marrow. Two γ-globin chains together with two α-globin chains constitute fetal hemoglobin (HbF) which is normally replaced by adult hemoglobin (HbA) in the year following birth (Higgs DR, Vickers MA, Wilkie AO, Pretorius IM, Jarman AP, Weatherall DJ (May 1989). “A review of the molecular genetics of the human alpha-globin gene cluster”. Blood. 73 (5): 1081-104.). The ENSEMBL IDs (i.e. the gene identifier number from the Ensembl Genome Browser database) for HBG1 and HBG2 are ENSG00000213934 and ENSG00000196565 respectively.

As used herein, the expression “increasing the fetal hemoglobin content” indicates that fetal hemoglobin is at least 5% higher in the eukaryotic cell treated with the DNA-targeting endonuclease, than in a comparable, eukaryotic cell, wherein an endonuclease targeting an unrelated locus is present or where no endonuclease is present. In some embodiments, the percentage of fetal hemoglobin expression in the eukaryotic cell is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than an eukaryotic cell. In some embodiments, any method known in the art can be used to measure an increase in fetal hemoglobin expression, e. g. HPLC analysis of fetal γ-globin protein and RT-qPCR analysis of fetal γ-globin mRNA. Typically, said methods are described in the EXAMPLE.

As used herein, the term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “promoter” has its general meaning in the art and refers to a nucleic acid sequence which is required for expression of a gene operably linked to the promoter sequence. HBG1 and HBG2 promoters are identical up to -221 bp and comprise the nucleic acid sequence as set forth in SEQ ID NO:1 and depicted in FIG. 1. According to the present invention, the first nucleotide in SEQ ID NO:1 denotes the nucleotide located at position -210 upstream of the HBG transcription starting site and the last nucleotide in SEQ ID NO:1 denotes the nucleotide located at position -100 upstream of the HBG transcription starting site. For instance,
the nucleotide at position -201 in the the HBG1 or HBG2 promoter denotes the nucleotide at position 10 in SEQ ID NO:1
the nucleotide at position -200 in the the HBG1 or HBG2 promoter denotes the nucleotide at position 11 in SEQ ID NO:1
the nucleotide at position -198 in the the HBG1 or HBG2 promoter denotes the nucleotide at position 13 in SEQ ID NO:1
the nucleotide at position -197 in the HBG1 or HBG2 promoter denotes the nucleotide at position 14 in SEQ ID NO:1,
the nucleotide at position -196 in the HBG1 or HBG2 promoter denotes the nucleotide at position 15 in SEQ ID NO:1, and,
the nucleotide at position -195 in the HBG1 or HBG2 promoter denotes the nucleotide at position 16 in SEQ ID NO:1.
the nucleotide at position -194 in the HBG1 or HBG2 promoter denotes the nucleotide at position 17 in SEQ ID NO:1.
the nucleotide at position -175 in the HBG1 or HBG2 promoter denotes the nucleotide at position 36 in SEQ ID NO:1.
the nucleotide at position -117 in the HBG1 or HBG2 promoter denotes the nucleotide at position 94 in SEQ ID NO:1.
the nucleotide at position -116 in the HBG1 or HBG2 promoter denotes the nucleotide at position 95 in SEQ ID NO:1.
the nucleotide at position -115 in the HBG1 or HBG2 promoter denotes the nucleotide at position 96 in SEQ ID NO:1.
the nucleotide at position -114 in the HBG1 or HBG2 promoter denotes the nucleotide at position 97 in SEQ ID NO:1.
the nucleotide at position -113 in the HBG1 or HBG2 promoter denotes the nucleotide at position 98 in SEQ ID NO:1.

SEQ ID NO 1:> Sequence of the HBG1 or HBG2 promoter

TTGGGGGCCCCTTCCCCACACTATCTCAATGCAAATATCTGTCTGAAACG
GTCCCTGGCTAAACTCCACCCATGGGTTGGCCAGCCTTGCCTTGACCAAT
AGCCTTGACAA

As used herein, the “-200 region” in the HBG1 or HBG2 promoter refers to the region which encompasses the nucleotides at position -197; -196 and -195 and thus relates to the region starting from the nucleotide at position 11 (i.e. -200) to the nucleotide at position 21 (i.e. -190) in SEQ ID NO:1, and more preferably to the region starting from the nucleotide at position 14 to the nucleotide at position 16 in SEQ ID NO:1.

As used herein, the “-115 region” in the HBG1 or HBG2 promoter refers to the region which encompasses the nucleotides at position -116, -115; -114 and -113 and thus relates to the region starting from the nucleotide at position 95 (i.e. -115) to the nucleotide at position 98 (i.e. -113) in SEQ ID NO:1

As used herein, the term “activator” refers to a transcriptional activator that is a protein (transcription factor) that increases gene transcription of a gene or set of genes. Most activators are DNA-binding proteins that bind to enhancers or promoter-proximal elements. According to the present disclosure, the activator is selected from the group consisting of KL1, TAL1 and GATA1.

As used herein, the term “transcriptional activator binding site” refers to a site present on DNA whereby the transcriptional activator according to the present disclosure binds. According to the present invention, the base-editing enzyme of the present invention edits the genome sequence of the eukaryotic cell so that the activator is able to bind to its transcriptional activator binding sites.

As used herein, the term “KLF1” has its general meaning in the art and refers to the Kruppel like factor 1 protein. The term is also known as EKLF; EKLF/KLF1. KLF1 is a hematopoietic-specific transcription factor that induces high-level expression of adult beta-globin and other erythroid genes. The zinc-finger protein binds to a DNA sequence found in the beta hemoglobin promoter.

As used herein, the term “TAL1” has its general meaning in the art and refers to the TAL bHLH transcription factor 1, erythroid differentiation factor. The term is also known as SCL; TCL5; tal-1; and bHLHa17.

As used herein, the term “GATA1” has its general meaning in the art and refers to the GATA binding protein 1. The term is also known as GF1; GF-1; NFE1; XLTT; ERYF1; NF-E1; XLANP; XLTDA; and GATA-1. GATA1 is a protein which belongs to the GATA family of transcription factors. The protein plays an important role in erythroid development by regulating the switch of fetal hemoglobin to adult hemoglobin.

As used herein, the term “repressor” refers to a transcriptional repressor that is a protein (transcription factor) that decreases gene transcription of a gene or set of genes. Most repressors are DNA-binding proteins that bind to enhancers or promoter-proximal elements. According to the present disclosure, the repressor is selected from the group consisting of BCL11A and LRF.

Accordingly, the term “transcriptional repressor binding site” refers to a site present on DNA whereby the transcription repressor binds. In some embodiments, the base-editing enzyme of the present invention edits the genome sequence of the eukaryotic cell so that the transcriptional repressor is not able to bind to its transcriptional repressor binding sites. In some embodiments, the DNA-targeting endonuclease of the present invention will inhibit the binding of LRF or BCL11A to its binding sites.

As used herein, the term “BCL11A” has its general meaning in the art and refers to the gene encoding for BAF chromatin remodeling complex subunit BCL11A (Gene ID: 53335). The term is also known as EVI9; CTIP1; DILOS; ZNF856; HBFQTL5; BCL11A-L; BCL11AS; BCL11a-M; or BCL11A-XL. Five alternatively spliced transcript variants of this gene, which encode distinct isoforms, have been reported. The protein associates with the SWI/SNF complex that regulates gene expression via chromatin remodelling. BCL11A is highly expressed in several hematopoietic lineages, and plays a role in the switch from γ- to β-globin expression during the fetal to adult transition (Sankaran VJ et al. “Human ƒetal hemoglobin expression is regulated by the developmental stage-specific repressor BCL11A ”, Science Science. 2008 Dec 19;322(5909):1839-42).

As used herein, the term “LRF” has its general meaning in the art and refers to the transcriptional repressor, which is Leukemia/lymphoma-related factor (LRF), encoded by the ZBTB7A gene. LRF is a ZBTB transcription factor that binds DNA through C-terminal C2H2-type zinc fingers and presumably recruits a transcriptional repressor complex through its N-terminal BTB domain (Lee SU, Maeda T. Immunol. Rev. 2012;247:107-119).

As used herein, the terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “nucleic acid molecule” or “polynucleotide” refers to a DNA molecule (for example, but not limited to, a cDNA or genomic DNA). The nucleic acid molecule can be single-stranded or double-stranded.

As used herein, the term “isolated” when referring to nucleic acid molecules or polypeptides means that the nucleic acid molecule or the polypeptide is substantially free from at least one other component with which it is associated or found together in nature.

As used herein, the term “complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, the term “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

As used herein, the term “hybridization” or “hybridizing” refers to a process where completely or partially complementary nucleic acid strands come together under specified hybridization conditions to form a double-stranded structure or region in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

As used herein, the term “base-editing enzyme” refers to fusion protein comprising a defective CRISPR/Cas nuclease linked to a deaminase polypeptide. The term is also known as “base-editor”. Two classes of base-editing enzymes--cytosine base-editing enzymes (CBEs) and adenine base-editing enzymes (ABEs)--can be used to generate single base pair edits without double stranded breaks. Typically, cytosine base-editing enzymes are created by fusing the defective CRISPR/Cas nuclease to a cytidine deaminase like APOBEC. Said base-editing enzymes are targeted to a specific locus by a gRNA, and they can convert cytidine to uridine within a small editing window near the PAM site. Uridine is subsequently converted to thymidine through base excision repair, creating a C to T change (or a G to A on the opposite strand.) Likewise, adenosine base-editing enzymes are created by fusing the defective CRISPR/Cas nuclease to an adenosine deaminase. Said base-editing enzymes have been engineered to convert adenosine to inosine, which is treated like guanosine by the cell, creating an A to G (or T to C) change.

As used herein, the term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains.

As used herein, the term “linker” refers to any means, entity or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

As used herein, the “linked” as used herein refers to the attachment of two or more entities to form one entity. A conjugate encompasses both peptide-small molecule conjugates as well as peptide-protein/peptide conjugates.

As used herein, the term “nuclease” includes a protein (i.e. an enzyme) that induces a break in a nucleic acid sequence, e.g., a single or a double strand break in a double-stranded DNA sequence.

As used herein, the term “CRISPR/Cas nuclease” has its general meaning in the art and refers to segments of prokaryotic DNA containing clustered regularly interspaced short palindromic repeats (CRISPR) and associated nucleases encoded by Cas genes. In bacteria the CRISPR/Cas loci encode RNA-guided adaptive immune systems against mobile genetic elements (viruses, transposable elements and conjugative plasmids). Three types of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR/Cas nucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activating small RNA (tracrRNA) that also serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG for S. Pyogenes Cas9) protospacer adjacent motif (PAM) to specify the cut site (the 3^{rd} or the 4^{th} nucleotide upstream from PAM).

As used herein, the term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′ -5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, the term “Cas9” refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI(NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1). Typically the Cas9 nuclease comprises the amino acid sequence as set forth in SEQ ID NO: 2.

SEQ ID NO:2: Cas9 sequence

MDKKYSIGLAIGTNSVGWAVITDEYKVPSKKFKVLGNTDRHSIKKNLIGA LLFDSGETAEATRLKRTA
RRRYTRRKNRICYLQEIFSNEMAKVDDSFFHR LEESFLVEEDKKHERHPIFGNIVDEVAYHEKYPTIY
HLRKKLVDSTDKAD LRLIYLALAHMIKFRGHFLIEGDLNPDNSDVDKLFIQLVQTYNQLFEENP INA
SGVDAKAILSARLSKSRRLENLIAQLPGEKKNGLFGNLIALSLGLTP NFKSNFDLAEDAKLQLSKDTY
DDDLDNLLAQIGDQYADLFLAAKNLSDAI LLSDILRVNTEITKAPLSASMIKRYDEHHQDLTLLKALV
RQQLPEKYKEI FFDQSKNGYAGYIDGGASQEEFYKFIKPILEKMDGTEELLVKLNREDLLR KQRTFD
NGSIPHQIHLGELHAILRRQEDFYPFLKDNREKIEKILTFRIPY YVGPLARGNSRFAWMTRKSEETIT
PWNFEEVVDKGASAQSFIERMTNFDK NLPNEKVLPKHSLLYEYFTVYNELTKVKYVTEGMRKPAFLSG
EQKKAIVD LLFKTNRKVTVKQLKEDYFKKIECFDSVEISGVEDRFNASLGTYHDLLKI IKDKDFLDN
EENEDILEDIVLTLTLFEDREMIEERLKTYAHLFDDKVMKQ LKRRRYTGWGRLSRKLINGIRDKQSGK
TILDFLKSDGFANRNFMQLIHDD SLTFKEDIQKAQVSGQGDSLHEHIANLAGSPAIKKGILQTVKVVD
ELVKV MGRHKPENIVIEMARENQTTQKGQKNSRERMKRIEEGIKELGSQILKEHP VENTQLQNEKLY
LYYLQNGRDMYVDQELDINRLSDYDVDHIVPQSFLKDD SIDNKVLTRSDKNRGKSDNVPSEEVVKKMK
NYWRQLLNAKLITQRKFDNL TKAERGGLSELDKAGFIKRQLVETRQITKHVAQILDSRMNTKYDENDK
LI REVKVITLKSKLVSDFRKDFQFYKVREINNYHHAHDAYLNAVVGTALIKK YPKLESEFVYGDYKV
YDVRKMIAKSEQEIGKATAKYFFYSNIMNFFKTEI TLANGEIRKRPLIETNGETGEIVWDKGRDFATV
RKVLSMPQVNIVKKTEV QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK YSLFELENGRKRMLASAG
ELQKGNELALPSKYVNFLYLASHYEKLKGSPE DNEQKQLFVEQHKHYLDEIIEQISEFSKRVILADAN
LDKVLSAYNKHRDK PIREQAENIIHLFTLTNLGAPAAFKYFDTTIDRKRYTSTKEVLDATLIHQ SIT
GLYETRIDLSQLGGD

As used herein, the term “defective CRISPR/Cas nuclease” refers to a CRISPR/Cas nuclease having lost at least one nuclease domain.

As used herein, the term “nickase” has its general meaning in the art and refers to an endonuclease which cleaves only a single strand of a DNA duplex. Accordingly, the term “Cas9 nickase” refers to a nickase derived from a Cas9 protein, typically by inactivating one nuclease domain of Cas9 protein.

As used herein, the term “deaminase” refers to an enzyme that catalyzes a deamination reaction. In some embodiments, the deaminase is a cytidine deaminase, catalyzing the hydrolytic deamination of cytidine or deoxycytidine to uracil or deoxyuracil, respectively. In some embodiments, the deaminase is an adenosine deaminase, catalyzing the hydrolytic deamination of adenosine to inosine, which is treated like guanosine by the cell, creating an A to G (or T to C) change.

As used herein, the term “guide RNA molecule” generally refers to an RNA molecule (or a group of RNA molecules collectively) that can bind to a Cas9 protein and target the Cas9 protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating RNA (tracrRNA).

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to using the CRISPR system as disclosed herein.

As used herein, the term “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “non-nuclease DNA modifying enzyme” refers to an enzyme that is not a nuclease but can introduce some modifications in a DNA molecule, such as a mutation.

As used herein, the term “cytidine deaminase” refers to enzyme that catalyzes the irreversible hydrolytic deamination of cytidine and deoxycytidine to uridine and deoxyuridine, respectively. The term “deamination”, as used herein, refers to the removal of an amine group from one molecule.

As used herein, the term “AID” or “Activation-Induced cytidine deaminase” refers to an enzyme that belongs to the APOBEC family of cytidine deaminase enzymes. AID is expressed within activated B cells and is required to initiate somatic hypermutation (Muramatsu et al., Cell, 102(5): 553-63 (2000); Revy et al., Cell, 102(5): 565-75 (2000); Yoshikawa et al., Science, 296(5575): 2033-6 (2002)) by creating point mutations in the underlying DNA encoding antibody genes (Martin et al., Proc. Natl. Acad. Sci. USA., 99(19): 12304-12308 (2002) and Nature, 415(6873): (2002); Petersen-Mart et al., Nature, 418(6893): 99-103 (2002)). AID is also an essential protein factor for class switch recombination and gene conversion (Muramatsu et al., Cell, 102(5): 553-63 (2000); Revy et al., Cell, 102(5): 565-75 (2000)).

As used herein, the term “ribonucleoprotein complex,” or “ribonucleoprotein particle” refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

As used herein the term “wild type” is a term of the art understood by skilled persons and means the typical form of an organism, strain, gene or characteristic as it occurs in nature as distinguished from mutant or variant forms.

As used herein, the term “mutation” has its general meaning in the art and refers to a substitution, deletion or insertion. The term “substitution” means that a specific amino acid residue at a specific position is removed and another amino acid residue is inserted into the same position. The term “deletion” means that a specific amino acid residue is removed. The term “insertion” means that one or more amino acid residues are inserted before or after a specific amino acid residue.

As used herein, the term “mutagenesis” refers to the introduction of mutations into a polynucleotide sequence. According to the present invention mutations are introduced into a target DNA molecule encoding for a variant domain of the antibody so as to mimic somatic hypermutation.

As used herein, the term “variant” refers to a first composition (e.g., a first molecule), that is related to a second composition (e.g., a second molecule, also termed a “parent” molecule). The variant molecule can be derived from, isolated from, based on or homologous to the parent molecule. A variant molecule can have entire sequence identity with the original parent molecule, or alternatively, can have less than 100% sequence identity with the parent molecule. For example, a variant of a sequence can be a second sequence that is at least 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100% identical in sequence compare to the original sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

As used herein, the term “derived from” refers to a process whereby a first component (e.g., a first molecule), or information from that first component, is used to isolate, derive or make a different second component (e.g., a second molecule that is different from the first).

As used herein, the term “therapeutically effective amount” is meant a sufficient amount of population of cells to treat the disease at a reasonable benefit/risk ratio applicable to any medical treatment. It will be understood that the total usage compositions of the present invention will be decided by the attending physician within the scope of sound medical judgment. The specific therapeutically effective dose level for any particular patient will depend upon a variety of factors including the age, body weight, general health, sex and diet of the patient, the time of administration, route of administration, the duration of the treatment, drugs used in combination or coincidental with the population of cells, and like factors well known in the medical arts. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer’s lactate can be utilized. The infusion medium can be supplemented with human serum albumin. A treatment-effective amount of cells in the composition is dependent on the relative representation of the cells with the desired specificity, on the age and weight of the recipient, and on the severity of the targeted condition. These amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. Typically, the minimal dose is 2 million of cells per kg. Usually 2 to 20 million of cells are injected in the subject. The desired purity can be achieved by introducing a sorting step. For uses provided herein, the cells are generally in a volume of a liter or less, can be 500 ml or less, even 250 ml or 100 ml or less. The clinically relevant number of cells can be apportioned into multiple infusions that cumulatively equal or exceed the desired total amount of cells.

### Methods

Accordingly, the first object of the present invention relates to a method for increasing fetal hemoglobin content in a eukaryotic cell comprising the step of contacting the eukaryotic cell with a gene editing platform that consists of a (a) at least one base-editing enzyme and (b) least one guide RNA molecule for guiding the base-editing enzyme to at least one target sequence in the HBG1 or HBG2 promoter, thereby editing said promoter and subsequently increasing the expression of gamma globin in said eukaryotic cell.

In some embodiments, the gene editing platform is suitable for introducing some mutations in the HBG1 or HBG2 promoter so that at least one transcriptional activator binding site is introduced in said promoter. In some embodiments, the gene editing platform is particularly suitable for introducing a new transcriptional activator binding site for KLF1, TAL1 or GATA1.

In some embodiments, the gene editing platform herein disclosed introduces the -198T>C mutation in the HBG1 or HBG2 promoter so that the KFL1 activator can now binds to the promoter.

In some embodiments, the gene editing platform herein disclosed introduces the -175T>C mutation in the HBG1 or HBG2 promoter so that the TAL1 activator can now binds to the promoter.

In some embodiments, the gene editing platform herein disclosed introduces the -113A>G mutation in the HBG1 or HBG2 promoter so that the GATA1 activator can now binds to the promoter.

In some embodiments, the gene editing herein disclosed is particularly suitable for editing the -200 region in the HBG1 or HBG2 promoter so that the binding site for the LRF repressor is disrupted. In some embodiments, the gene editing platform herein disclosed introduces at least one mutation selected from the group consisting of -201C>T, -200C>T, -197C>T, -196C>T, -195C>T and -194C>T in the HBG1 or HBG2 promoter so that the binding site for the LRF repressor is disrupted.

In some embodiments, the gene editing herein disclosed is particularly suitable for editing the -115 region in the HBG1 or HBG2 promoter so that the binding site for the BCL11A repressor is disrupted. In some embodiments, the gene editing platform herein disclosed introduces at least one mutation selected from the group consisting of -114C>T, -113C>T, -115C>T and -116C>T in the HBG1 or HBG2 promoter so that the binding site for the BCL1 1A repressor is disrupted.

In some embodiments, the eukaryotic cell is selected from the group consisting of hematopoietic progenitor cells, hematopoietic stem cells (HSCs), pluripotent cells (i.e. embryonic stem cells (ES) and induced pluripotent stem cells (iPS)). Typically, the eukaryotic cell results from a stem cell mobilization.

In some embodiments, the base-editing enzyme of the present invention comprises a defective CRISPR/Cas nuclease. The sequence recognition mechanism is the same as for the non-defective CRISPR/Cas nuclease. Typically, the defective CRISPR/Cas nuclease of the invention comprises at least one RNA binding domain. The RNA binding domain interacts with a guide RNA molecule as defined hereinafter. However the defective CRISPR/Cas nuclease of the invention is a modified version with no nuclease activity. Accordingly, the defective CRISPR/Cas nuclease specifically recognizes the guide RNA molecule and thus guides the base-editing enzyme to its target DNA sequence.

In some embodiments, the defective CRISPR/Cas nuclease can be modified to increase nucleic acid binding affinity and/or specificity, alter an enzymatic activity, and/or change another property of the protein. In some embodiments, the nuclease domains of the protein can be modified, deleted, or inactivated. In some embodiments, the protein can be truncated to remove domains that are not essential for the function of the protein. In some embodiments, the protein is truncated or modified to optimize the activity of the RNA binding domain.

In some embodiments, the CRISPR/Cas nuclease consists of a mutant CRISPR/Cas nuclease i.e. a protein having one or more point mutations, insertions, deletions, truncations, a fusion protein, or a combination thereof. In some embodiments, the mutant has the RNA-guided DNA binding activity, but lacks one or both of its nuclease active sites. In some embodiments, the mutant comprises an amino acid sequence having at least 50% of identity with the wild type amino acid sequence of the CRISPR/Cas nuclease. Various CRISPR/Cas nucleases can be used in this invention. Non-limiting examples of suitable CRISPR/CRISPR/Cas nucleases include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g., WO2014144761 WO2014144592, WO2013176772, US20140273226, and US20140273233, the contents of which are incorporated herein by reference in their entireties.

In some embodiments, the CRISPR/Cas nuclease is derived from a type II CRISPR-Cas system. In some embodiments, the CRISPR/Cas nuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammoniƒex degensii, Caldicelulosiruptor becscii, Candidatus Desulƒorudis, Clostridium botulinum, Clostridium difficile, Finegoldia magna, Natranaerobius thermophilus, Pelotomaculum thermopropionicum, Acidithiobacillus caldus, Acidithiobacillus ƒerrooxidans, Allochromatium vinosum, Marinobacter sp., Nitrosococcus halophilus, Nitrosococcus watsoni, Pseudoalteromonas haloplanktis, Ktedonobacter racemiƒer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho aƒricanus, or Acaryochloris marina, inter alia.

In some embodiments, the CRISPR/Cas nuclease is a mutant of a wild type CRISPR/Cas nuclease (such as Cas9) or a fragment thereof. In some embodiments, the CRISPR/Cas nuclease is a mutant Cas9 protein from S. pyogenes.

Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H841A completely inactivate the nuclease activity of S. pyogenes Cas9 (Jinek et al., Science. 337:816-821(2012); Qi et al., Cell. 28; 152(5):1173-83 (2013).

In some embodiments, the CRISPR/Cas nuclease of the present invention is nickase and more particularly a Cas9 nickase i.e. the Cas9 from S. pyogenes having one mutation selected from the group consisting of D10A and H840A. In some embodiments, the nickase of the present invention comprises the amino acid sequence as set forth in SEQ ID NO: 3 or SEQ ID NO:33.

SEQ ID NO: 3> S. pyogenes nCas9 Protein Sequence having the D10A mutation

MDKKYSIGLA IGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

SEQ ID NO: 33> S. pyogenes nCas9 Protein Sequence having the H840A mutation

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDA IVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In some embodiments, the Cas9 variants having mutations other than D10A or H840A are used, which e.g., result in nuclease inactivated Cas9 (dCas9). Such mutations, by way of example, include other amino acid substitutions at D10 and H840, or other substitutions within the nuclease domains of Cas9 (e.g., substitutions in the HNH nuclease subdomain and/or the RuvC1 subdomain). In some embodiments, variants of dCas9 are provided which are at least about 70% identical, at least about 80% identical, at least about 90% identical, at least about 95% identical, at least about 98% identical, at least about 99% identical, at least about 99.5% identical, or at least about 99.9% to SEQ ID NO: 2 or 3. In some embodiments, variants of dCas9 are provided having amino acid sequences which are shorter, or longer than SEQ ID NO: 2 or 3, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

According to the present invention, the second component of the base-editing enzyme herein disclosed comprises a non-nuclease DNA modifying enzyme that is a deaminase.

In some embodiments, the deaminase is a cytidine deaminase. In some embodiments, the deaminase is an apolipoprotein B mRNA-editing complex (APOBEC) family deaminase. In some embodiments, the deaminase is an APOBEC1 family deaminase. In some embodiments, the deaminase is an activation-induced cytidine deaminase (AID). In some embodiments, the deaminase is an ACF1/ASE deaminase.

In some embodiments, the deaminase is selected from the group consisting of AID: activation induced cytidine deaminase, APOBEC1: apolipoprotein B mRNA editing enzyme, catalytic polypeptide-like 1, APOBEC3A: apolipoprotein B mRNA editing enzyme, catalytic polypeptide-like 3A, APOBEC3B: apolipoprotein B mRNA editing enzyme, catalytic polypeptide-like 3B, APOBEC3C: apolipoprotein B mRNA editing enzyme, catalytic polypeptide-like 3C, APOBEC3D: apolipoprotein B mRNA editing enzyme, catalytic polypeptide-like 3D, APOBEC3F: apolipoprotein B mRNA editing enzyme, catalytic polypeptide-like 3F, APOBEC3G: apolipoprotein B mRNA editing enzyme, catalytic polypeptide-like 3G, APOBEC3H: apolipoprotein B mRNA editing enzyme, catalytic polypeptide-like 3H, ADA: adenosine deaminase, ADAR1: adenosine deaminase acting on RNA 1, Dnmt1: DNA (cytosine-5-)-methyltransferase 1, Dnmt3a: DNA (cytosine-5-)-methyltransferase 3 alpha, Dnmt3b: DNA (cytosine-5-)-methyltransferase 3 beta and Tet1: methylcytosine dioxygenase.

In some embodiments, the deaminase derives from the Activation Induced cytidine Deaminase (AID). AID is a cytidine deaminase that can catalyze the reaction of deamination of cytosine in the context of DNA or RNA. When brought to the targeted site, AID changes a C base to U base. In dividing cells, this could lead to a C to T point mutation. Alternatively, the change of C to U could trigger cellular DNA repair pathways, mainly excision repair pathway, which will remove the mismatching U-G base-pair, and replace with a T-A, A-T, C-G, or G-C pair. As a result, a point mutation would be generated at the target C-G site. In some embodiments, the DNA modifying enzyme is AID*Δ that is an AID mutant with increased SHM activity whose Nuclear Export Signal (NES) has been removed (Hess GT, Fresard L, Han K, Lee CH, Li A, Cimprich KA, Montgomery SB, Bassik MC: Directed evolution using dCas9-targeted somatic hypermutation in mammalian cells. Nat Methods 2016, 13(12):1036-1042).

In some embodiments, the deaminase is an adenosine deaminase. In some embodiments, the deaminase is an ADAT family deaminase. For example, an ADAT family adenosine deaminase can be fused to a Cas9 domain, e.g., a nuclease-inactive Cas9 domain, thus yielding a Cas9-ADAT fusion protein.

In some embodiments, the deaminase consists of a variant of the amino acid sequence as set forth in SEQ ID NO:4-14.

SEQ ID NO:4 Human AID:

MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL

SEQ ID NO:5 Human APOBEC-3G

MKPHFRNTVERMYRDTFSYNFYNRPILSRRNTVWLCYEVKTKGPSRPPLD
AKIFRGQVYSELKYHPEMRFFHWFSKWRKLHRDQEYEVTWYISWSPCTKC
TRDMATFLAEDPKVTLTIFVARLYYFWDPDYQEALRSLCQKRDGPRATMK
IMNYDEFQHCWSKFVYSQRELFEPWNNLPKYYILLHIMLGEILRHSMDPP
TFTFNFNNEPWVRGRHETYLCYEVERMHNDTWVLLNQRRGFLCNQAPHKH
GFLEGRHAELCFLDVIPFWKLDLDQDYRVTCFTSWSPCFSCAQEMAKFIS
KNKHVSLCIFTARIYDDQGRCQEGLRTLAEAGAKISIMTYSEFKHCWDTF
VDHQGCPFQPWDGLDEHSQDLSGRLRAILQNQEN

SEQ ID NO:6 Human APOBEC-3F

MKPHFRNTVERMYRDTFSYNFYNRPILSRRNTVWLCYEVKTKGPSRPRLD
AKIFRGQVYSQPEHHAEMCFLSWFCGNQLPAYKCFQITWFVSWTPCPDCV
AKLAEFLAEHPNVTLTISAARLYYYWERDYRRALCRLSQAGARVKIMDDE
EFAYCWENFVYSEGQPFMPWYKFDDNYAFLHRTLKEILRNPMEAMYPHIF
YFHFKNLRKAYGRNESWLCFTMEVVKHHSPVSWKRGVFRNQVDPETHCHA
ERCFLSWFCDDILSPNTNYEVTWYTSWSPCPECAGEVAEFLARHSNVNLT
IFTARLYYFWDTDYQEGLRSLSQEGASVEIMGYKDFKYCWENFVYNDDEP
FKPWKGLKYNFLFLDSKLQEILE

SEQ ID NO: 7 Human APOBEC-3B:

MNPQIRNPMERMYRDTFYDNFENEPILYGRSYTWLCYEVKIKRGRSNLLW
DTGVFRGQVYFKPQYHAEMCFLSWFCGNQLPAYKCFQITWFVSWTPCPDC
VAKLAEFLSEHPNVTLTISAARLYYYWERDYRRALCRLSQAGARVTIMDY
EEFAYCWENFVYNEGQQFMPWYKFDENYAFLHRTLKEILRYLMDPDTFTF
NFNNDPLVLRRRQTYLCYEVERLDNGTWVLMDQHMGFLCNEAKNLLCGFY
GRHAELRFLDLVPSLQLDPAQIYRVTWFISWSPCFSWGCAGEVRAFLQEN
THVRLRIFAARIYDYDPLYKEALQMLRDAGAQVSIMTYDEFEYCWDTFVY
RQGCPFQPWDGLEEHSQALSGRLRAILQNQGN

SEQ ID NO:8 Human APOBEC-3C:

MNPQIRNPMKAMYPGTFYFQFKNLWEANDRNETWLCFTVEGIKRRSVVSW
KTGVFRNQVDSETHCHAERCFLSWFCDDILSPNTKYQVTWYTSWSPCPDC
AGEVAEFLARHSNVNLTIFTARLYYFQYPCYQEGLRSLSQEGVAVEIMDY
EDFKYCWENFVYNDNEPFKPWKGLKTNFRLLKRRLRESLQ

SEQ ID NO:9 Human APOBEC-3A:

MEASPASGPRHLMDPHIFTSNFNNGIGRHKTYLCYEVERLDNGTSVKMDQ
HRGFLHNQAKNLLCGFYGRHAELRFLDLVPSLQLDPAQIYRVTWFISWSP
CFSWGCAGEVRAFLQENTHVRLRIFAARIYDYDPLYKEALQMLRDAGAQV
SIMTYDEFKHCWDTFVDHQGCPFQPWDGLDEHSQALSGRLRAILQNQGN

SEQ ID NO: 10 Human APOBEC-3H:

MALLTAETFRLQFNNKRRLRRPYYPRKALLCYQLTPQNGSTPTRGYFENK
KKCHAEICFINEIKSMGLDETQCYQVTCYLTWSPCSSCAWELVDFIKAHD
HLNLGIFASRLYYHWCKPQQKGLRLLCGSQVPVEVMGFPKFADCWENFVD
HEKPLSFNPYKMLEELDKNSRAIKRRLERIKIPGVRAQGRYMDILCDAEV

SEQ ID NO:11 Human APOBEC-3D

MNPQIRNPMERMYRDTFYDNFENEPILYGRSYTWLCYEVKIKRGRSNLLW
DTGVFRGPVLPKRQSNHRQEVYFRFENHAEMCFLSWFCGNRLPANRRFQI
TWFVSWNPCLPCVVKVTKFLAEHPNVTLTISAARLYYYRDRDWRWVLLRL
HKAGARVKIMDYEDFAYCWENFVCNEGQPFMPWYKFDDNYASLHRTLKEI
LRNPMEAMYPHIFYFHFKNLLKACGRNESWLCFTMEVTKHHSAVFRKRGV
FRNQVDPETHCHAERCFLSWFCDDILSPNTNYEVTWYTSWSPCPECAGEV
AEFLARHSNVNLTIFTARLCYFWDTDYQEGLCSLSQEGASVKIMGYKDFV
SCWKNFVYSDDEPFKPWKGLQTNFRLLKRRLREILQ

SEQ ID NO:12 Human APOBEC-1:

MTSEKGPSTGDPTLRRRIEPWEFDVFYDPRELRKEACLLYEIKWGMSRKI
WRSSGKNTTNHVEVNFIKKFTSERDFHPSMSCSITWFLSWSPCWECSQAI
REFLSRHPGVTLVIYVARLFWHMDQQNRQGLRDLVNSGVTIQIMRASEYY
HCWRNFVNYPPGDEAHWPQYPPLWMMLYALELHCIILSLPPCLKISRRWQ
NHLTFFRLHLQNCHYQTIPPHILLATGLIHPSVAWR

SEQ ID NO:13 Human ADAT-2:

MEAKAAPKPAASGACSVSAEETEKWMEEAMHMAKEALENTEVPVGCLMVY
NNEVVGKGRNEVNQTKNATRHAEMVAIDQVLDWCRQSGKSPSEVFEHTVL
YVTVEPCIMCAAALRLMKIPLVVYGCQNERFGGCGSVLNIASADLPNTGR
PFQCIPGYRAEEAVEMLKTFYKQENPNAPKSKVRKKECQKS

SEQ ID NO:14 Human ADAT-1:

MWTADETAQLCYEHYGIRLPKKGKPEPNHEWTLLAAVVKIQSPADKACDT
PDKPVQVTKEVVSMGTGTKCIGQSKMRKNGDILNDSHAEVIARRSFQRYL
LHQLQLAATLKEDSIFVPGTQKGVWKLRRDLIFVFFSSHTPCGDASIIPM
LEFEDQPCCPVFRNWAHNSSVEASSNLEAPGNERKCEDPDSPVTKKMRLE
PGTAAREVTNGAAHHQSFGKQKSGPISPGIHSCDLTVEGLATVTRIAPGS
AKVIDVYRTGAKCVPGEAGDSGKPGAAFHQVGLLRVKPGRGDRTRSMSCS
DKMARWNVLGCQGALLMHLLEEPIYLSAVVIGKCPYSQEAMQRALIGRCQ
NVSALPKGFGVQELKILQSDLLFEQSRSAVQAKRADSPGRLVPCGAAISW
SAVPEQPLDVTANGFPQGTTKKTIGSLQARSQISKVELFRSFQKLLSRIA
RDKWPHSLRVQKLDTYQEYKEAASSYQEAWSTLRKQVFGSWIRNPPDYHQ
FK

In some embodiments, the deaminase is fused to the N-terminus of the defective CRISPR/Cas nuclease. In some embodiments, the deaminase is fused to the C-terminus of the defective CRISPR/Cas nuclease. In some embodiments, the defective CRISPR/Cas nuclease and the deaminase are fused via a linker. In some embodiments, the linker comprises a (GGGGS)n (SEQ ID NO:3), a (G)n, an (EAAAK)n (SEQ ID NO: 4), a (GGS)n, an SGSETPGTSESATPES (SEQ ID NO: 5) motif (see, e.g., Guilinger J P, Thompson D B, Liu D R. Additional suitable linker motifs and linker configurations will be apparent to those of skill in the art. In some embodiments, suitable linker motifs and configurations include those described in Chen et al., Fusion protein linkers: property, design and functionality. Adv Drug Deliv Rev. 2013; 65(10):1357-69, the entire contents of which are incorporated herein by reference.

In some embodiments, the fusion protein may comprise additional features. Other exemplary features that may be present are localization sequences, such as nuclear localization sequences (NLS), cytoplasmic localization sequences, export sequences, such as nuclear export sequences, or other localization sequences, as well as sequence tags that are useful for solubilization, purification, or detection of the fusion proteins. Suitable localization signal sequences and sequences of protein tags are provided herein, and include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable features will be apparent to those of skill in the art.

Various base-editing enzymes are known in the art (see e.g. Improving cytidine and adenine base-editing enzymes by expression optimization and ancestral reconstruction. Nat Biotechnol. 2018 May 29) and typically include those described in Table A.

TABLE A

some exemplary base-editing enzymes

| | |
|---|---|
| Base-editing enzyme | References |
| ABEmax | Improving cytidine and adenine base-editing enzymes by expression optimization and ancestral reconstruction. Nat Biotechnol. 2018 May 29. pii: nbt.4172. doi: 10.1038/nbt.4172. |
| AncBE4max | Improving cytidine and adenine base-editing enzymes by expression optimization and ancestral reconstruction. Nat Biotechnol. 2018 May 29. pii: nbt.4172. doi: 10.1038/nbt.4172. |
| evoCDA1-BE4max-NG | Continuous evolution of base-editing enzymes with expanded target compatibility and improved activity. Nat Biotechnol. 2019 Jul 22. pii: 10.1038/s41587-019-0193-0. doi: 10.1038/s41587-019-0193-0. |
| evoFERNY-BE4max | Continuous evolution of base-editing enzymes with expanded target compatibility and improved activity. Nat Biotechnol. 2019 Jul 22. pii: 10.1038/s41587-019-0193-0. doi: 10.1038/s41587-019-0193-0. |
| CBE-NRCH | Miller SM, Wang T, Randolph PB, Arbab M, Shen MW, Huang TP, Matuszek Z, Newby GA, Rees HA, Liu DR. Continuous evolution of SpCas9 variants compatible with non-G PAMs. Nat Biotechnol. 2020 Apr;38(4):471-481. doi: 10.1038/s41587-020-0412-8. Epub 2020 Feb 10. PMID: 32042170; PMCID: PMC7145744. |
| CBE-SpG | Walton RT, Christie KA, Whittaker MN, Kleinstiver BP. Unconstrained genome targeting with near-PAMless engineered CRISPR-Cas9 variants. Science. 2020 Apr 17;368(6488):290-296. doi: 10.1126/science.aba8853. Epub 2020 Mar 26. PMID: 32217751; PMCID: PMC7297043. |
| CBE-SpRY | Walton RT, Christie KA, Whittaker MN, Kleinstiver BP. Unconstrained genome targeting with near-PAMless engineered CRISPR-Cas9 variants. Science. 2020 Apr 17;368(6488):290-296. doi: 10.1126/science.aba8853. Epub 2020 Mar 26. PMID: 32217751; PMCID: PMC7297043. |
| ABE8e | Richter MF, Zhao KT, Eton E, Lapinaite A, Newby GA, Thuronyi BW, Wilson C, Koblan LW, Zeng J, Bauer DE, Doudna JA and Liu DR. Phage-assisted evolution of an adenine base editor with improved Cas domain compatibility and activity. Nat. Biotechnol. 2020 Mar 38, 883-891. 1780 doi: 10.1038/s41587-020-0453-z |

The second component of the gene-editing platform disclosed herein consists of at least one guide RNA molecule suitable for guiding the base-editing enzyme to at least one target sequence located in the HBG1 or HBG2 promoter. The guide RNA molecule of the present invention thus comprises a guide sequence for providing the targeting specificity. It includes a region that is complementary and capable of hybridization to a pre-selected target site of interest in the HBG1 or HBG2 promoter.

In some embodiment, this guide sequence can comprise from about 10 nucleotides to more than about 25 nucleotides. For example, the region of base pairing between the guide sequence and the corresponding target site sequence can be about 10, 11, 12, 13, 14, 15, 16, 17, 18, 19, 20, 22, 23, 24, 25, or more than 25 nucleotides in length. In some embodiments, the guide sequence is about 17-20 nucleotides in length, such as 20 nucleotides.

Typically, a software program is used to identify candidate CRISPR target sequences on both strands of the DNA nucleic acid molecule containing the HBG genes based on desired guide sequence length and a CRISPR motif sequence (PAM) for a specified CRISPR enzyme. One requirement for selecting a suitable target nucleic acid is that it has a 3′ PAM site/sequence. Each target sequence and its corresponding PAM site/sequence are referred herein as a Cas-targeted site. Type II CRISPR system, one of the most well characterized systems, needs only Cas 9 protein and a guide RNA complementary to a target sequence to affect target cleavage. For example, target sites for Cas9 from S. pyogenes, with PAM sequences NGG, may be identified by searching for 5′-Nx-NGG-3′ both on the input sequence and on the reverse-complement of the input. Since multiple occurrences in the genome of the DNA target site may lead to nonspecific genome editing, after identifying all potential sites, the program filters out sequences based on the number of times they appear in the relevant reference genome. For those CRISPR enzymes for which sequence specificity is determined by a “seed” sequence, such as the 11-12 bp 5′ from the PAM sequence, including the PAM sequence itself, the filtering step may be based on the seed sequence. Thus, to avoid editing at additional genomic loci, results are filtered based on the number of occurrences of the seed:PAM sequence in the relevant genome. The user may be allowed to choose the length of the seed sequence. The user may also be allowed to specify the number of occurrences of the seed:PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s). Further details of methods and algorithms to optimize sequence selection can be found in U.S. Appln. Ser. No. 61/836,080; incorporated herein by reference.

In some embodiments, the base-editing enzyme and the corresponding guide RNA molecule is chosen according to Table B.

TABLE B

suitable pairings between base-editing enzymes and guide RNA molecule

| | | | |
|---|---|---|---|
| Goal / Target | Selected base-editing enzyme | Selected gRNA molecule | gRNA name |
| Creation of binding site for KLF1 and TAL1 | ABEmax | AUAUUUGCAUUGAGAUAGUG (TAL1) (SEQ ID NO:48) | TAL1_bs_1 |
| | | | KLF1_bs_1 |
| | | GUGGGGAAGGGGCCCCCAAG (KLF1) (SEQ ID NO:49) | |
| Disruption of the BCL11A binding site | AncBE4max | CUUGACCAAUAGCCUUGACA (SEQ ID NO :50) | BCL11A_bs_1 |
| | ABEmax | CUUGACCAAUAGCCUUGACA (SEQ ID NO :50) | BCL11A_bs_1 |
| | evoCDA1-BE4max-NG | 1: CUUGACCAAUAGCCUUGACA (SEQ ID NO :50) | BCL11A_bs_1 |
| | | | BCL11A_bs_2 |
| | | 4: UUGACCAAUAGCCUUGACAAGG (SEQ ID NO :51) | |
| | evoFERNY-BE4max-NG | 1: CUUGACCAAUAGCCUUGACA (SEQ ID NO :50) | BCL11A_bs_1 |
| | | | BCL11A_bs_2 |
| | | 4: UUGACCAAUAGCCUUGACAA (SEQ ID NO :51) | |
| Disruption of the LRF binding site | evoCDA1-BE4max-NG | CCUUCCCCACACUAUCUCAA (SEQ ID NO :52) | LRF_bs_1 |
| | evoFERNY-BE4max-NG | CCUUCCCCACACUAUCUCAA (SEQ ID NO :52) | LRF_bs_1 |
| | AncBE4max_NAA | GCCCCUUCCCCACACUAUCU (SEQ ID NO :53) | LRF_bs_2 |
| | CBE-NRCH | CCUUCCCCACACUAUCUCAA (SEQ ID NO :52) | LRF_bs_1 |
| | CBE-SpG | CCUUCCCCACACUAUCUCAA (SEQ ID NO :52) | LRF_bs_1 |
| | CBE-SpRY | CCUUCCCCACACUAUCUCAA (SEQ ID NO :52) | LRF_bs_1 |
| | CBE-SpRY | GCCCCUUCCCCACACUAUCU (SEQ ID NO :53) | LRF_bs_2 |
| | ABE8e | GUGGGGAAGGGGCCCCCAAG (KLF1) (SEQ ID NO:49) | KLF1_bs_1 |

The guide RNA molecule of the present invention can be made by various methods known in the art including cell-based expression, in vitro transcription, and chemical synthesis. The ability to chemically synthesize relatively long RNAs (as long as 200 mers or more) using TC-RNA chemistry (see, e.g., U.S. Pat. No. 8,202,983) allows one to produce RNAs with special features that outperform those enabled by the basic four ribonucleotides (A, C, G and U). In particular, the RNA molecule of the present invention can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties.

In some embodiments, the guide RNA molecule may include one or more modifications. Such modifications may include inclusion of at least one non-naturally occurring nucleotide, or a modified nucleotide, or analogs thereof. Modified nucleotides may be modified at the ribose, phosphate, and/or base moiety. Modified nucleotides may include 2′-O-methyl analogs, 2′-deoxy analogs, or 2′-fluoro analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, pseudouridine, inosine, 7-methylguanosine.

In some embodiments, a plurality of guide RNA molecules are designed for targeting a plurality of sequences in the HBG1 or HBG2 promoter. In some embodiments, the gene editing platform disclosed herein thus comprises 2, 3, 4, 5, 6, 7, 8, 9, 10, 11, 12, 13, 14, 15, 16, 17, 18, or 20 guide RNA molecules as disclosed herein.

In some embodiments, a plurality of base-editing enzyme along with a plurality of guide RNA molecules are designed for targeting a plurality of sequences in the HBG1 or HBG2 promoter. In some embodiments, the gene editing platform disclosed herein thus comprises 2, 3 or 4 base-editing enzymes and 2, 3, 4, 5, 6, 7, 8, 9, 10, 11, 12, 13, 14, 15, 16, 17, 18, or 20 RNA molecules as disclosed herein.

In some embodiments, the different components of the gene editing platform of the present invention are provided to the eukaryotic cell through expression from one or more expression vectors. For example, the nucleic acids encoding the guide RNA molecule or the base-editing enzyme can be cloned into one or more vectors for introducing them into the eukaryotic cell. The vectors are typically prokaryotic vectors, e.g., plasmids, or shuttle vectors, or insect vectors, for storage or manipulation of the nucleic acid encoding the guide RNA molecule or the base-editing enzyme herein disclosed. Preferably, the nucleic acids are isolated and/or purified. Thus, the present invention provides recombinant constructs or vectors having sequences encoding one or more of the guide RNA molecule or base-editing enzymes described above. Examples of the constructs include a vector, such as a plasmid or viral vector, into which a nucleic acid sequence of the invention has been inserted, in a forward or reverse orientation. In some embodiments, the construct further includes regulatory sequences. A “regulatory sequence” includes promoters, enhancers, and other expression control elements (e.g., polyadenylation signals). Regulatory sequences include those that direct constitutive expression of a nucleotide sequence, as well as inducible regulatory sequences. The design of the expression vector can depend on such factors as the choice of the eukaryotic cell to be transformed, transfected, or infected, the desired expression level, and the like. Large numbers of suitable vectors and promoters are known to those of skill in the art, and are commercially available. Appropriate cloning and expression vectors for use with eukaryotic hosts are also described in e.g., Sambrook et al. (2001, Molecular Cloning: A Laboratory Manual, Cold Spring Harbor Press). The vector can be capable of autonomous replication or integration into a host DNA. The vector may also include appropriate sequences for amplifying expression. In addition, the expression vector preferably contains one or more selectable marker genes to provide a phenotypic trait for selection of transformed host cells such as dihydrofolate reductase or neomycin resistance for eukaryotic cell cultures, or such as tetracycline or ampicillin resistance in E. coli. Any of the procedures known in the art for introducing foreign nucleotide sequences into host cells may be used. Examples include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell.

In some embodiments, the different components of the gene editing platform of the present invention are provided to the population of cells through the use of an RNA-encoded system. For instance, the base-editing system may provided to the population of cells through the use of a chemically modified mRNA-encoded adenine or cytidine base editor together with modified guide RNA as described in Jiang, T., Henderson, J.M., Coote, K. et al. Chemical modifications of adenine base editor mRNA and guide RNA expand its application scope. Nat Commun 11, 1979 (2020). In particular, engineered RNA-encoded base-editing enzymes (e.g. ABE) system are prepared by introducing various chemical modifications to both mRNA that encoded the base-editing enzyme and guide RNA. In particular said modifications consist in uridine depleted mRNAs modified with 5-methoxyuridine: synonymous codons may be introduced to deplete uridines as much as possible without altering the coding sequence and replaced all the remaining uridines with 5-methoxyuridine. Said optimized base editing system exhibits higher editing efficiency at some genomic sites compared to DNA-encoded system. It is also possible to encapsulate the modified mRNA and guide RNA into lipid nanoparticle (LNP) for allowing lipid nanoparticle (LNP)-mediated delivery.

In some embodiments, the different components of the gene editing platform of the present invention are provided to the population of cells through the use of ribonucleoprotein (RNP) complexes. For instance the base-editing enzyme can be pre-complexed with one or more guide RNA molecules to form a ribonucleoprotein (RNP) complex. The RNP complex can thus be introduced into the eukaryotic cell. Introduction of the RNP complex can be timed. The cell can be synchronized with other cells at G1, S, and/or M phases of the cell cycle. RNP delivery avoids many of the pitfalls associated with mRNA, DNA, or viral delivery. Typically, the RNP complex is produced simply by mixing the proteins (i.e. the base-editing enzyme) and one or more guide RNA molecules in an appropriate buffer. This mixture is incubated for 5-10 min at room temperature before electroporation. Electroporation is a delivery technique in which an electrical field is applied to one or more cells in order to increase the permeability of the cell membrane. In some embodiments, genome editing efficiency can be improved by adding a transfection enhancer oligonucleotide.

In some embodiments, a plurality of successive transfections are performed for reaching a desired level of mutagenesis in the cell.

A further object of the present invention relates to a method for increasing fetal hemoglobin levels in a subject in need thereof, the method comprising transplanting a therapeutically effective amount of a population of eukaryotic cells obtained by the method as above described.

In some embodiments, the population of cell is autologous to the subject, meaning the population of cells is derived from the same subject.

In some embodiments, the subject has been diagnosed with a hemoglobinopathy. The method of the present invention is thus particularly suitable for the treatment of hemoglobinopathies.

In some embodiments, the β-hemoglobinopathy is a sickle cell disease.

In some embodiments, the hemoglobinopathy is a β-thalassemia.

### Kits

This invention further provides kits containing reagents for performing the above-described methods, including all component of the gene editing platform as disclosed herein for performing mutagenesis. To that end, one or more of the reaction components, e.g., guide RNA molecules, and nucleic acid molecules encoding for the base-editing enzymes for the methods disclosed herein can be supplied in the form of a kit for use. In some embodiments, the kit comprises one or more base-editing enzymes and one or more guide RNA molecules. In some embodiments, the kit can include one or more other reaction components. In some embodiments, an appropriate amount of one or more reaction components is provided in one or more containers or held on a substrate. Examples of additional components of the kits include, but are not limited to, one or more host cells, one or more reagents for introducing foreign nucleotide sequences into host cells, one or more reagents (e.g., probes or PCR primers) for detecting expression of the guide RNA or base-editing enzymes or verifying the target nucleic acid’s status, and buffers or culture media for the reactions. The kit may also include one or more of the following components: supports, terminating, modifying or digestion reagents, osmolytes, and an apparatus for detection. The components used can be provided in a variety of forms. For example, the components (e.g., enzymes, RNAs, probes and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits containing protein components or complexes thereof in a liquid, it is preferred that they are provided and maintained below 0° C., preferably at or below -20° C., or otherwise in a frozen state. The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

The invention will be further illustrated by the following figures and examples. However, these examples and figures should not be interpreted in any way as limiting the scope of the present invention.

### Figures

FIG. 1: HPFH mutations in the HBG½ promoters, in the β-globin locus. Schematic representation of the β-globin locus on chromosome 11, depicting the HBG2 and HBG1 genes and their promoters. The sequence of the HBG2 and HBG1 identical promoters (from -214 to -98 nucleotides upstream of the HBG TSS) is shown below. Black arrows indicate HPFH mutations described at HBG1 and/or HBG2 promoters. HPFH mutations that lead to the de novo creation of transcriptional activator (TAL1, KLF1 and GATA1) binding sites, are above the DNA sequence, while HPFH mutations that lead to the disruption of transcriptional repressor (LRF and BCL11A) binding sites are below the DNA sequence. HPFH mutations that can be generated by base editing are highlighted with rectangles. Ovals indicate transcriptional activators and repressors. Target sequences of the gRNAs designed to be used with base editing enzymes, are reported in the bottom part of the figure (highlighted with dark arrows) and aligned with the DNA sequence that they bind to. The target bases are highlighted in white bold and the rest of the protospacer sequence is highlighted in grey. The PAM (protospacer adjacent motif) for each gRNA is reported in black at the end of the arrows.

FIG. 2: Efficient base editing of the HBG½ promoters in K562 cells. (A-M) A-T to G-C (A-C, N) or C-G to T-A (D-M) base editing efficiency, calculated by the EditR software in samples subjected to Sanger sequencing. The base editing efficiency percentage was measured by subtracting the percentage of the base conversion in the control that was considered as background noise. On the top of each graph the target and the enzyme used are indicated. Data are expressed as mean±SD (n=2-3 biologically independent experiments). (O) The frequency of insertions and deletions (InDel), measured by TIDE analysis, is reported for the control and the base edited samples. (P) The frequency of the 4.9-kb deletion, measured by ddPCR, is reported for control and base edited samples, and for a positive control (DNA extracted from K562 cells edited with the canonical Cas9 nuclease). (Q) Binding site conversion after base editing as analyzed by DiffLogo⁽²⁶⁾. *Two different gRNAs (BCL11A_bs_1 and BCL11A_bs_2) were used in combination with the enzymes evoCDA1-BE4max-NG and evoFERNY-BE4max-NG (graphs E and F). Similarly, two different gRNAs (LRF_bs_1 and LRF_bs_2) were used in combination with CBE-SpRY enzyme (graphs L and M).

FIG. 3: Efficient base editing of the HBG½ promoters in HUDEP-2 cells. A-T to G-C (A-C) or C-G to T-A (D-H) base editing efficiency, calculated by the EditR software in samples subjected to Sanger sequencing. The base editing efficiency was calculated as described in FIG. 2 legend. On the top of each graph, the target and the enzyme used are indicated. (I) The frequency of insertions and deletions (InDel), measured by TIDE analysis, is reported for the control and the base edited samples. (J) Binding site conversion after base editing as analyzed by DiffLogo⁽²⁶⁾. *Two different gRNAs (BCL11A_bs_1 and BCL11A_bs_2) were used in combination with the enzyme evoCDA1-BE4max-NG and evoFERNY-BE4max-NG (graphs E and F).

FIG. 4: HbF de-repression after generation of the TAL1 and KLF1 binding sites in HUDEP-2 cells. (A) Frequency of HbF-expressing cells (as determined by flow cytometry), in Glycophorin A (GPA)^{high} populations at day 0 (D0) and day 9 (D9) of erythroid differentiation. The base editing efficiency is indicated on the top of each black bar (D0). (B) RT-qPCR analysis of β- and γ-globin mRNA levels at D0, 6 and 9 of erythroid differentiation. β- and γ-globin mRNA expression was normalized to α-globin mRNA and expressed as percentage of the total β-like globins. (C). Expression of γ- (^{G}γ- + ^{A}γ-) and β-globin chains measured by RP-HPLC. β-like globin chain expression was normalized to α-globin. (D) Analysis of HbF and HbA by cation-exchange HPLC. We calculated the percentage of each Hb type over the total Hb tetramers. (E-G) Flow-cytometry analysis of the late erythroid marker GYPA (E) and of the early erythroid markers CD36 (F) and CD71 (G) at D0 and D9 of HUDEP-2 differentiation.

FIG. 5: HbF reactivation upon disruption of the BCL11A binding site in HUDEP-2 cells. (A-C) Flow-cytometry analysis of the late erythroid marker GYPA (A) and of the early erythroid markers CD36 (B) and CD71 (C) at D0 and D9 of HUDEP-2 differentiation. (D) Frequency of HbF-expressing cells (as determined by flow cytometry), in GPA^{high} populations at day 0 (D0) and day 9 (D9) of erythroid differentiation. The base editing efficiency is indicated on the top of each black bar (D0). (E) RT-qPCR analysis of β- and γ-globin mRNA levels at D0, 6 and 9 of erythroid differentiation. β- and γ-globin mRNA expression was normalized to α-globin mRNA and expressed as percentage of the total β-like globins. (F) Expression of γ-(^{G}γ- + ^{A}γ-) and β-globin chains measured by RP-HPLC. β-like globin expression was normalized to α-globin. (G) Analysis of HbF and HbA by cation-exchange HPLC. We calculated the percentage of each Hb type over the total Hb tetramers.

FIG. 6: HbF increase upon disruption of the LRF binding site in HUDEP-2 and HSPCs. (A) Frequency of HbF-expressing cells (as determined by flow cytometry) in GPA^{high} populations in undifferentiated HUDEP-2 cells. (B) To edit the LRF binding site, HSPCs were transfected with two plasmids expressing the AncBE4max_NAA and the LRF_bs_1 gRNA, respectively. Base editing efficiency in the LRF binding site, as calculated by the EditR software in HSPC samples subjected to Sanger sequencing. The base editing efficiency percentage was calculated as described in FIG. 2 legend. The frequency of insertions and deletions (InDel), measured by TIDE analysis, is reported for the control and the base edited samples. (C) Binding site conversion after base editing in HSPCs as analyzed by DiffLogo⁽²⁶⁾. (D) Quantification of HbF and HbA by cation-exchange HPLC in a bulk population of control and HBG-edited BFU-E colonies. We plotted the percentage of each Hb type over the total Hb tetramers. Control cells include samples transfected either with TE buffer or with the AncBE4max_NAA plasmid only, or with the AncBE4max_NAA plasmid and a gRNA targeting an unrelated to the β-globin locus site (AAVS1 site; Weber et al., Sc. Advances, 2020).

FIG. 7: Base editing efficiency by enzymes with low RNA off-target activity, in the HBG½ promoters, in K562 cells. A-T to G-C (A-C) or C-G to T-A (D) base editing efficiency, calculated by the EditR software in samples subjected to Sanger sequencing. The base editing efficiency percentage was calculated as described in FIG. 2 legend. On the top of each graph, the target and the enzyme used are indicated. For each target, we compared the classical base editing enzyme and the base editing enzyme with lower RNA off-target activity.

FIG. 8: Testing CBEs to disrupt the LRF binding site in SCD HSPCs. C-G to T-A (A and B) base editing efficiency, calculated by the EditR software in samples subjected to Sanger sequencing. (C) The frequency of insertions and deletions (InDel), measured by TIDE analysis, is reported for base edited samples. (D) RT-qPCR analysis of β^{S}- and γ-globin mRNA levels at Day 13 of erythroid differentiation. β^{S}- and γ-globin mRNA expression was normalized to α-globin mRNA and expressed as percentage of the total β-like globins. (E) Expression of γ- (^{G}γ-+ ^{A}γ-) and β^{S}-globin chains measured by RP-HPLC. β-like globin chain expression was normalized to α-globin. (F) Analysis of HbF and HbS by cation-exchange HPLC. We calculated the percentage of each Hb type over the total Hb tetramers. (G) Frequency of HbF-expressing cells (as determined by flow cytometry), in Glycophorin A (GPA)^{high} populations at Day 19 of erythroid differentiation. (H) Frequency of non-sickle cells upon O₂ deprivation in mock-transfected control and base edited samples. (D-F and H) Below each graph, the base editing efficiency (BE%), the percentage of HbF over the total Hb tetramers (HbF%), as measured by cation-exchange HPLC and the frequency of HbF-expressing cells, as determined by flow cytometry (F-cells%) are indicated for each sample. Donor number n=1.

FIG. 9: Testing ABEs to disrupt the LRF binding site or create the KLF1 binding site in SCD HSPCs. A-T to G-C (A and D) base editing efficiency, calculated by the EditR software in samples subjected to Sanger sequencing. (E) The frequency of insertions and deletions (InDel), measured by TIDE analysis, is reported for base edited samples. (F) RT-qPCR analysis of β^{s}- and γ-globin mRNA levels at Day 13 of erythroid differentiation. β^{S}- and γ-globin mRNA expression was normalized to α-globin mRNA and expressed as percentage of the total β-like globins. (G) Expression of γ- (^{G}γ- + ^{A}γ-) and β^{S}-globin chains measured by RP-HPLC. β-like globin chain expression was normalized to α-globin. (H) Analysis of HbF and HbS by cation-exchange HPLC. We calculated the percentage of each Hb type over the total Hb tetramers. (I) Frequency of non-sickle cells upon O₂ deprivation in control (mock-transfected and edited in an unrelated AAVSI1 locus) and base edited samples. (J) Frequency of HbF-expressing cells (as determined by flow cytometry), in Glycophorin A (GPA)^{high} populations at Day 19 of erythroid differentiation. (F-I) Below each graph, the base editing efficiency (BE%), the percentage of HbF over the total Hb tetramers (HbF%), as measured by cation-exchange HPLC and the frequency of HbF-expressing cells, as determined by flow cytometry (F-cells%) are indicated for each sample. Donor number n=1.

FIG. 10: RNA-mediated base editing at the HBG½ promoters in K562 cells and SCD HSPCs. (A-H) A-T to G-C or C-G to T-A base editing efficiency in K562 and HSPCs. The base editing efficiency was calculated by the EditR software in K562 (A-E) and SCD HSPC (F-H) samples subjected to Sanger sequencing. On the top of each graph the enzyme and the gRNA used are indicated.

### EXAMPLE

### Methods

### Cell Line Culture

K562 were maintained in RPMI 1640 (Lonza) containing glutamine and supplemented with 10% fetal bovine serum (Lonza), 2 mM Hepes (Life Technologies), 100 nM sodium pyruvate (Life Technologies), and penicillin and streptomycin (Life Technologies). HUDEP-2 cells were cultured in StemSpan SFEM (Stem Cell Technologies), supplemented with 1 µg/mL doxycycline (Sigma), 10⁻⁶ M dexamethasone (Sigma), 100 ng/mL human stem cell factor (SCF) (Peprotech), 3IU/mL erythropoietin (Necker Hospital Pharmacy), L-glutamine (Life Technologies) and penicillin/streptomycin. HUDEP-2 cells were differentiated in Iscove’s Modified Dulbecco’s Medium (IMDM) (Life Technologies) supplemented with 330 µg/mL holo-transferrin (Sigma), 10 µg/mL recombinant human insulin (Sigma), 2IU/mL heparin (Sigma), 5% human AB serum, 3IU/mL erythropoietin, 100 ng/mL human SCF, 1 µg/mL doxycycline, 1% L-glutamine, and 1% penicillin/streptomycin for 9 days. Flow cytometry analysis of CD36, CD71 and GYPA surface markers and a standard May-Grumwald Giemsa staining were performed to monitor erythroid differentiation.

### HSPC Purification and Culture

We obtained human cord blood (CB) CD34⁺ HSPCs from healthy donors and human non-mobilized peripheral blood CD34⁺ HSPCs from SCD patients. CB samples eligible for research purposes were obtained because of a convention with the CB bank of Saint Louis Hospital (Paris, France). SCD samples eligible for research purposes were obtained because of a convention with “Hôpital Necker-Enfants malades” Hospital (Paris, France). Written informed consent was obtained from all adult subjects. All experiments were performed in accordance with the Declaration of Helsinki. The study was approved by the regional investigational review board (reference: DC 2014-2272, CPP Ile-de-France II “Hôpital Necker-Enfants malades”). HSPCs were purified by immunomagnetic selection with AutoMACS (Miltenyi Biotec) after immunostaining with the CD34 MicroBead Kit (Miltenyi Biotec). Forty-eight hours before transfection, CD34⁺ cells (10⁶cells/ml) were thawed and cultured in the “HSPC medium” containing StemSpan (STEMCELL Technologies) supplemented with penicillin/streptomycin (Gibco), 250 nM StemRegenin1 (STEMCELL Technologies), and the following recombinant human cytokines (PeproTech): human stem cell factor (SCF) (300 ng/ml), Flt-3L (300 ng/ml), thrombopoietin (TPO) (100 ng/ml), and interleukin-3 (IL-3) (60 ng/ml).

### Plasmids

Plasmids used in this study include pCMV_ABEmax_P2A_GFP (Addgene #112101), pCMV_AncBE4max_P2A_GFP (Addgene #112100), pBT374 (Addgene #125615), pBT372 (Addgene #125613), pCMV-ABEmaxAW (Addgene #125647), pMJ920 (Addgene #42234), ABE8e (Addgene #138489), pCMV-BE4max-NRCH (Addgene #136920), pCAG-CBE4max-SpG-P2A-EGFP (RTW4552) (Addgene #139998) and pCAG-CBE4max-SpRY-P2A-EGFP (RTW5133) (Addgene #139999). The AncBE4max_NAA plasmid was created by replacing the PAM interaction domain of the SpCas9n with the one of the SmaCas9, while the plasmid AncBE4max_R33A/K34A was created by inserting the mutations R33A and K34A in the APOBEC1 domain of the AncBE4max plasmid (Addgene #112094). A DNA fragment (3′UTR+poly-A) containing two copies of the 3′ untranslated region (UTR) of the HBB gene and a poly-A sequence of 96 adenines was purchased by Genscript (gene synthesis). Similarly, a DNA fragment containing the uridine depleted coding sequence of pCAG-CBE4max-SpRY-P2A-EGFP was generated (CBE-SpRY_U-delp). The CBE-SpRY-OPT plasmid was generated by inserting the 3′UTR+poly-A fragment in pCAG-CBE4max-SpRY-P2A-EGFP and by replacing CBE4max-SpRY with the CBE-SpRY_U-delp fragment. Furthermore, we replaced the CAG synthetic promoter of CBE-SpRY plasmid with a T7 promoter. The ABE-SpRY-OPT plasmid was created by inserting the 3′UTR+poly-A fragment in pCMV-T7-ABEmax(7.10)- SpRY-P2A-EGFP (RTW5025) (Addgene #140003).

### gRNA Design

For the gRNA expression plasmid construction, oligonucleotides were annealed to create the gRNA protospacer and the duplexes were ligated into Bbs I-digested MA128 plasmid (provided by M. Amendola, Genethon, France).

TABLE 1

gRNA target sequences.

| gRNA | Target sequence (5′ to 3′) | Position (hg19) | Strand |
|---|---|---|---|
| TAL1_bs_1 | ATATTTGCATTGAGATAGTGTGG (SEQ ID NO : 15) | chr11: 5271260-5271279 (HBG1) | + |
| | | chr11: 5276184-5276203 (HBG2) | |
| KLF1_bs_1 | GTGGGGAAGGGGCCCCCAAGAGG (SEQ ID NO : 16) | chr11: 5271279-5271298 (HBG1) | + |
| | | chr11: 5276203-5276222 (HBG2) | |
| BCL11A_bs_1 | CTTGACCAATAGCCTTGACAAGG (SEQ ID NO : 17) | chr11: 5271188-5271207 (HBG1) | - |
| | | chr11: 5276112-5276131 (HBG2) | |
| BCL11_Abs_2 | TTGACCAATAGCCTTGACAAGG (SEQ ID NO 18) | chr11: 5271187-5271206 (HBG1) | - |
| | | chr11: 5276111-5276130 (HBG2) | |
| LRF_bs_1 | CCTTCCCCACACTATCTCAATG (SEQ ID NO 19) | chr11: 5271269-5271288 (HBG1) | - |
| | | chr11: 5276193-5276212 (HBG2) | |
| LRF_bs_2 | GCCCCTTCCCCACACTATCTCAA (SEQ ID NO :20) | chr11: 5271272-5271291 (HBG1) | - |
| | | chr11: 5276196-5276215 (HBG2) | |

PAM motif is highlighted in bold.

### mRNA in Vitro Transcription

10 µg of CBE-SpRY-OPT, ABE-SpRY-OPT or ABE8e expressing plasmids were digested overnight with 20 Units of a restriction enzyme that cuts once right after the poly-A tail (AflII for CBE-SpRY and ABE-SpRY and SapI for ABE8e). The linearized plasmids were purified using a PCR purification kit (QIAGEN #28106) and were eluted in 30 µl of DNase/RNase-free water. 1 µg of linearized plasmid was used as template for the in vitro transcription reaction (MEGAscript, Ambion #AM1334). The in vitro transcription protocol was modified as follows.

The GTP nucleotide solution was used at a final concentration of 3.0 mM instead of 7.5 mM and the anti-reverse cap analog N7-Methyl-3′-O-Methyl-Guanosine-5′-Triphosphate-5′-Guanosine (ARCA, Trilink #N-7003) was used at a final concentration of 12.0 mM resulting in a final ratio of Cap:GTP of 4:1 that allows efficient capping of the mRNA. The incubation time for the in vitro reaction was reduced to 30 minutes. After DNaseI treatment (MEGAscript, Ambion #AM1334), the ABE8e mRNA was poly-A tailed according to the manufacturer’s protocol (Poly-A tailing kit, Ambion #AM1350). mRNA was precipitated using lithium chloride and resuspended in TE buffer in a final volume that allowed to achieve a concentration of ≥ 1 µg/µl. The mRNA quality was checked using Bioanalyzer (Agilent).

### Plasmid Transfection

K562 and HUDEP-2 cells (10⁶ cells/condition) were transfected with 3.6 µg of a base editing enzyme expressing plasmid and 1.2 µg of gRNA-containing plasmid. For base editing enzyme plasmids that do not expressing GFP, we co-transfected 250 ng of a GFPmax expressing plasmid (Lonza). We used AMAXA Cell Line Nucleofector Kit V (VCA-1003) and U-16 and L-29 programs (Nucleofector II) for K562 and HUDEP-2, respectively. For K562 cells, transfection efficiency was evaluated by flow cytometry, using the Fortessa X20 (BD Biosciences) flow cytometer 18 h after transfection and cells were maintained in culture for at least 3 days and at day 3 genomic DNA extraction was performed. GFP⁺ HUDEP-2 cells were sorted 18h after transfection using SH800 Cell Sorter (Sony Biotechnology) and sorted cells were expanded in culture. 3 days after transfection, genomic DNA extraction was performed. CD34⁺ HSPCs (10⁶ cells/condition) were transfected with 3.6 µg of the enzyme-expressing plasmid and 2.4 µg of the gRNA-containing plasmid. For base editing enzyme plasmids that do not express GFP, we co-transfected 250 ng of a GFPmax expressing plasmid (Lonza). We used AMAXA Human CD34 Cell Nucleofector Kit (VPA-1003) and U-08 program (Nucleofector II). 18 h after transfection, GFP⁺ CD34⁺ HSPCs were sorted using SH800 Cell Sorter (Sony Biotechnology) and either plated at a concentration of 500,000/mL in cytokine-enriched HSPC medium (described above) for at least 6 days and then genomic DNA extraction was performed, or were differentiated in mature RBCs using a 3-phase erythroid differentiation protocol (Weber, Frati Science Advances 2020) of up to 20 days. Genomic DNA extraction was performed at Day 6, total RNA extraction was performed at Day 13 and functional analyses to evaluate the HbF expression (flow cytometry, RP-HPLC, CE-HPLC and a sickling assay) were conducted at day 19.

### RNA Transfection

K562 cells (2×10⁵ cells/condition) were transfected with 2.0 µg of a base editor-expressing mRNA and a synthetic gRNA containing chemical modifications (2′-O-Methyl at 3 first and last bases, 3′ phosphorothioate bonds between first 3 and last 2 bases) purchased from Synthego, at a final concentration of 1.5 µM. We used the P3 Primary Cell 4D- Nucleofector X Kit S (Lonza) and the CA137 program (Nucleofector 4D). Cells transfected with TE buffer served as negative control. RNA-transfected K562 cells were maintained in culture for at least 3 days prior to genomic DNA extraction and base editing analysis.

CD34⁺ HSPCs (2×10⁵ cells/condition) were transfected with 3.0 µg of a base editor-expressing mRNA and a synthetic gRNA containing chemical modifications (2′-O-Methyl at 3 first and last bases, 3′ phosphorothioate bonds between first 3 and last 2 bases) purchased from Synthego, at a final concentration of 4.6 µM. We used the P3 Primary Cell 4D- Nucleofector X Kit S (Lonza) and the CA137 program (Nucleofector 4D). Cells transfected with TE buffer or with the base editor mRNA only, or with the base editor mRNA and a gRNA targeting the AAVS1 locus, served as negative controls. RNA-transfected HSPCs were plated at a concentration of 500,000/mL in the HSPC medium (described above) and cultured for at least 6 days prior to genomic DNA extraction and base editing analysis.

### Evaluation of Editing Efficiency

Genomic DNA was extracted from control and edited cells using PURE LINK Genomic DNA Mini kit (LifeTechnologies) following manufacturer’s instructions, 3 days post-transfection for K562 and HUDEP2 cells and 6 days post-trasnfection for CD34⁺ HSPCs. To evaluate base editing efficiency at gRNA target sites, we performed PCR followed by Sanger sequencing and EditR analysis (EditR: A Method to Quantify Base Editing from Sanger Sequencing)⁽²⁷⁾. TIDE analysis (Tracking of InDels by Decomposition) was also performed in order to evaluate the percentage of insertion and deletion (InDels) in base edited samples⁽²⁸⁾.

TABLE 2

Primers used to detect base editing and InDels events.

| | | |
|---|---|---|
| Amplified region | F/R | Sequence (5′ to 3′) |
| HBG1 + HBG2 promoters | F | AAAAACGGCTGACAAAAGAAGTCCTGGT AT (SEQ ID NO :21) |
| | R | ATAACCTCAGACGTTCCAGAAGCGAGTGT G (SEQ ID NO :22) |

Digital Droplet PCR (ddPCR) was performed using EvaGreen mix or primer/probe mix (Biorad) to quantify the frequency of the 4.9-kb deletion by amplifying the HBG1-HBG2 intervening region I or II respectively. Short (~1 min) elongation time allowed the PCR amplification of the genomic region harboring the deletion. Control primers annealing to a genomic region on the same chromosome (chr 11) or to hALB (chr 4) were used as DNA loading control respectively.

TABLE 3

Primers used for ddPCR.

| Amplified region | F/R | Sequence (5′ to 3′) |
|---|---|---|
| HBG1-HBG2 intervening region I | F | GTTTTAAAACAACAAAAATGAGGGAAA GA (SEQ ID NO :23) |
| | R | GTTGCTTTATAGGATTTTTCACTACAC (SEQ ID NO :24) |
| Chr11 control region | F | CCCTTCCGAGAGGATTTAGG (SEQ ID NO :25) |
| | R | AGTCGGGATCTGAACAATGG (SEQ ID NO :26) |
| HBG1-HBG2 intervening region II | F | ACGGATAAGTAGATATTGAGGTAAGC (SEQ ID NO :54) |
| | R | GTCTCTTTCAGTTAGCAGTGG (SEQ ID NO :55) |
| hALB | F | ACTCATGGGAGCTGCTGGTT (SEQ ID NO :56) |
| | R | GCTGTCATCTCTTGTGGGCTG SEQ ID NO : 57) |

F, forward primer; R, reverse primer.

### Flow Cytometry Analysis

Differentiated HUDEP-2 were fixed and permeabilized using BD Cytofix/Cytoperm solution (BD Pharmingen) and stained with an antibody recognizing HbF (APC-conjugated anti HbF antibody, MHF05, Life Technologies). Flow cytometry analysis of CD36, CD71 and GYPA erythroid surface markers was performed using a V450-conjugated anti-CD36 antibody (561535, BD Horizon), a FITC-conjugated anti-CD71 antibody (555536, BD Pharmingen) and a PE-Cy7-conjugated anti- GYPA antibody (563666, BD Pharmingen). SCD RBCs differentiated from control and edited HSPCs were fixed with 0.05% glutaraldehyde. permeabilized with 0.1% TRITON X-100 and stained with an antibody recognizing HbF (FITC-conjugated anti HbF antibody, clone 2D12 552829 BD). Flow cytometry analysis of CD36, CD71, GYPA, BAND3 and α4-Integrin erythroid surface markers was performed using a V450-conjugated anti-CD36 antibody (561535, BD Horizon), a FITC-conjugated anti-CD71 antibody (555536, BD Pharmingen), a PE-Cy7-conjugated anti-GYPA antibody (563666, BD Pharmingen), a PE-cpnjugated anti-BAND3 antibody (9439, IBGRL) and a APC-conjugated anti-CD49d antibody (559881, BD). Flow cytometry analysis of DRAQ5 (enucleation) and 7AAD (viability) was performed using anti-double stranded DNA dyes (65-0880-96, Invitrogen and 559925, BD, respectively). Flow cytometry analyses were performed using Fortessa X20 (BD Biosciences) or Gallios flow cytometers. Data were analyzed using the FlowJo (BD Biosciences) or KALUZA software.

### Colony-Forming Cell (CFC) Assay

HSPCs were plated at a concentration of 1×10³ cells/mL in methylcellulose-containing medium (GFH4435, Stem Cell Technologies) under conditions supporting erythroid and granulomonocytic differentiation. BFU-E and CFU-GM colonies were scored after 14 days. BFU-Es and CFU-GMs were randomly picked and collected as bulk populations (containing at least 30 colonies) to evaluate the hemoglobin expression by CE-HPLC.

### RT-qPCR Analysis of Globin Transcripts

Total RNA was extracted from HUDEP-2 (at day 0, 6 and 9 of differentiation) and erythroid cells differentiated from SCD HSPCs (at day 13) using RNeasy micro kit (QIAGEN), following manufacturer’s instructions. Mature transcripts were reverse-transcribed using SuperScript First-Strand Synthesis System for RT-qPCR (Invitrogen) with oligo (dT) primers. RT-qPCR was performed using iTaq universal SYBR Green master mix (Biorad) and a Viia7 Real-Time PCR system (ThermoFisher Scientific).

TABLE 4

Primers used for RT-qPCR.

| Amplified region | F/R | Sequence (5′ to 3′) |
|---|---|---|
| HBA | F | CGGTCAACTTCAAGCTCCTAA (SEQ ID NO :27) |
| | R | ACAGAAGCCAGGAACTTGTC (SEQ ID NO :28) |
| HBB | F | GCAAGGTGAACGTGGATGAAGT (SEQ ID NO :29) |
| | R | TAACAGCATCAGGAGTGGACAGA (SEQ ID NO :30) |
| HBG1+HBG2 | F | CCTGTCCTCTGCCTCTGCC (SEQ ID NO :31) |
| | R | GGATTGCCAAAACGGTCAC (SEQ ID NO :32) |

F, forward primer; R, reverse primer.

### RP-HPLC Analysis of Globin Chains

RP-HPLC analysis was performed using a NexeraX2 SIL-30AC chromatograph and the LC Solution software (Shimadzu). Globin chains were separated by HPLC using a 250×4.6 mm, 3.6 µm Aeris Widepore column (Phenomenex). Samples were eluted with a gradient mixture of solution A (water/acetonitrile/trifluoroacetic acid, 95:5:0.1) and solution B (water/acetonitrile/trifluoroacetic acid, 5:95:0.1). The absorbance was measured at 220 nm.

### CE-HPLC Analysis of Hemoglobin Tetramers

Cation-exchange HPLC analysis was performed using a NexeraX2 SIL-30AC chromatograph and the LC Solution software (Shimadzu). Hemoglobin tetramers were separated by HPLC using a 2 cation-exchange column (PolyCAT A, PolyLC, Columbia, MD). Samples were eluted with a gradient mixture of solution A (20 mM bis Tris, 2 mM KCN, pH=6.5) and solution B (20 mM bis Tris, 2 mM KCN, 250 mM NaCl, pH=6.8). The absorbance was measured at 415 nm.

### Sickling Assay

At the end of the erythroid differentiation, mature RBCs derived from SCD HSPCs were incubated under hypoxia conditions (0% O₂) and the time course of sickling was monitored in real time by video microscopy, capturing images every 20 min for at least 60 min using an AxioObserver Z1 microscope (Zeiss) and a 40× objective. Images of the same fields were taken throughout all stages and processed with ImageJ to determine the percentage of non-sickle RBCs per field of acquisition in the total RBC population. More than 400 cells were counted per condition.

### Results

### Efficient Base Editing in the HBG Promoters Leads to the De Novo Generation of Binding Sites for TAL1 and KLF1 Transcriptional Activators

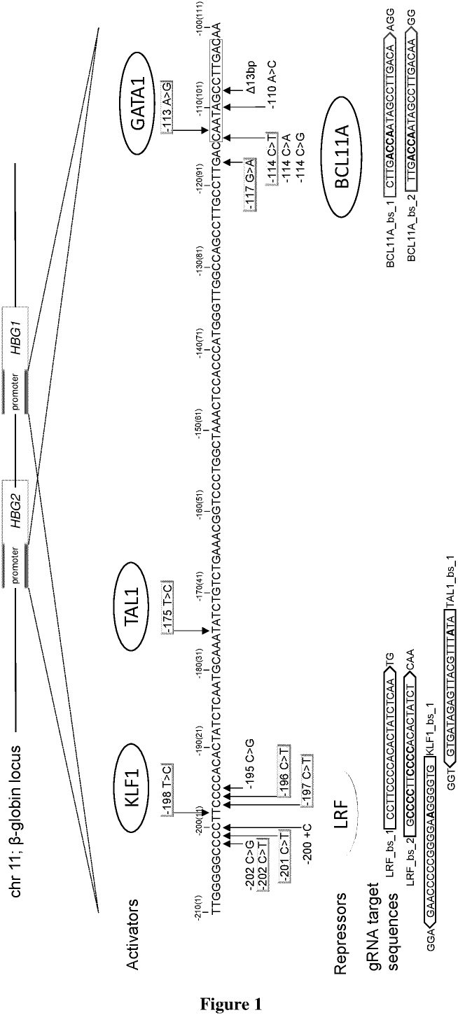

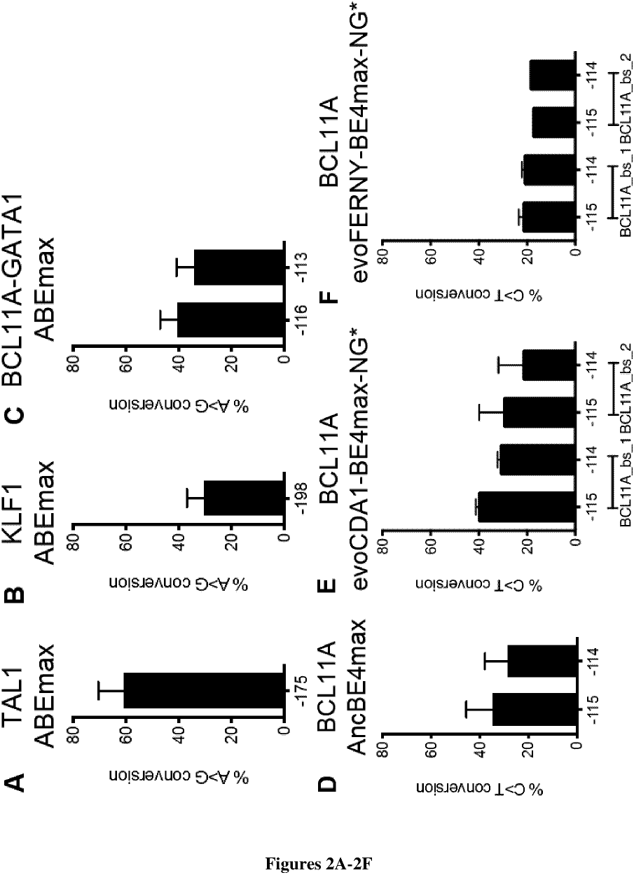

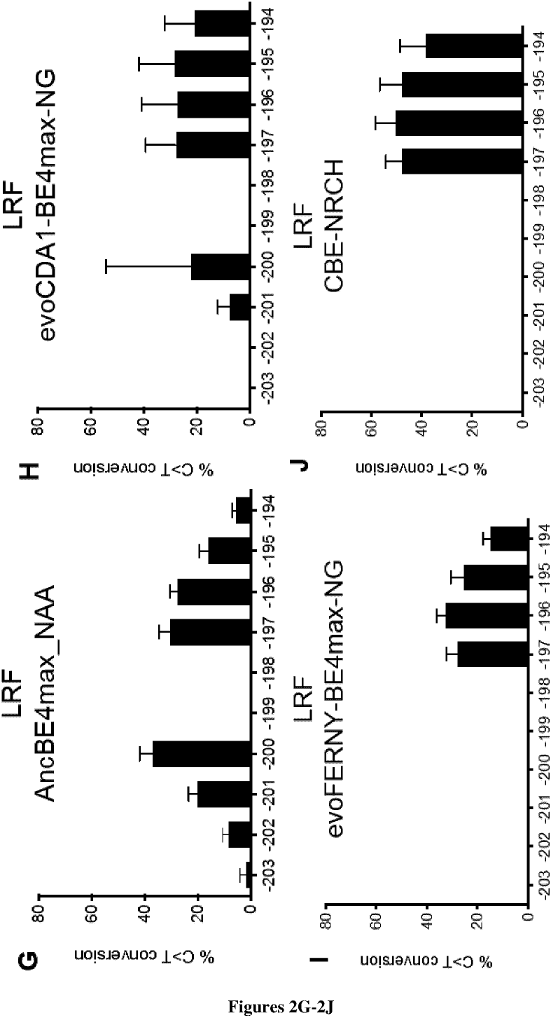

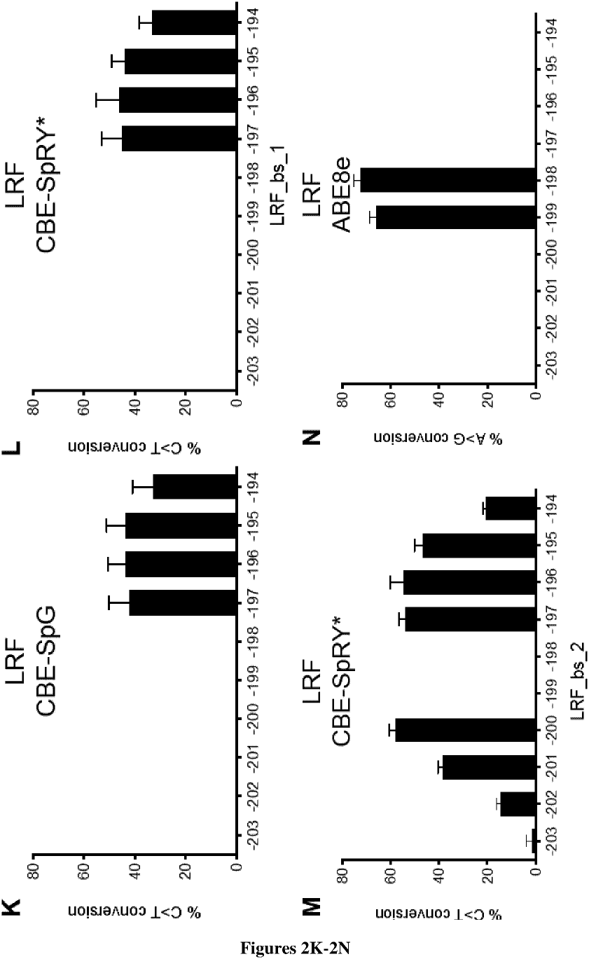

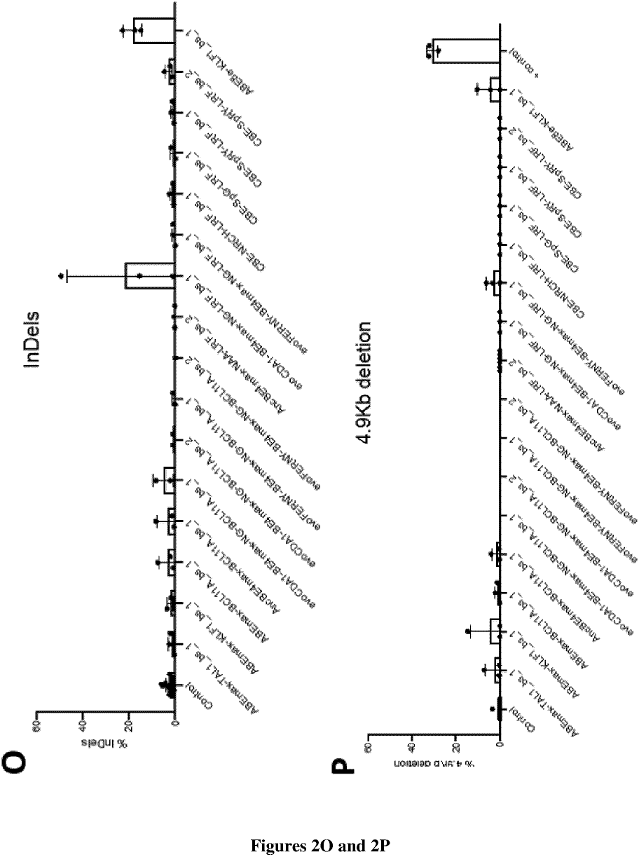

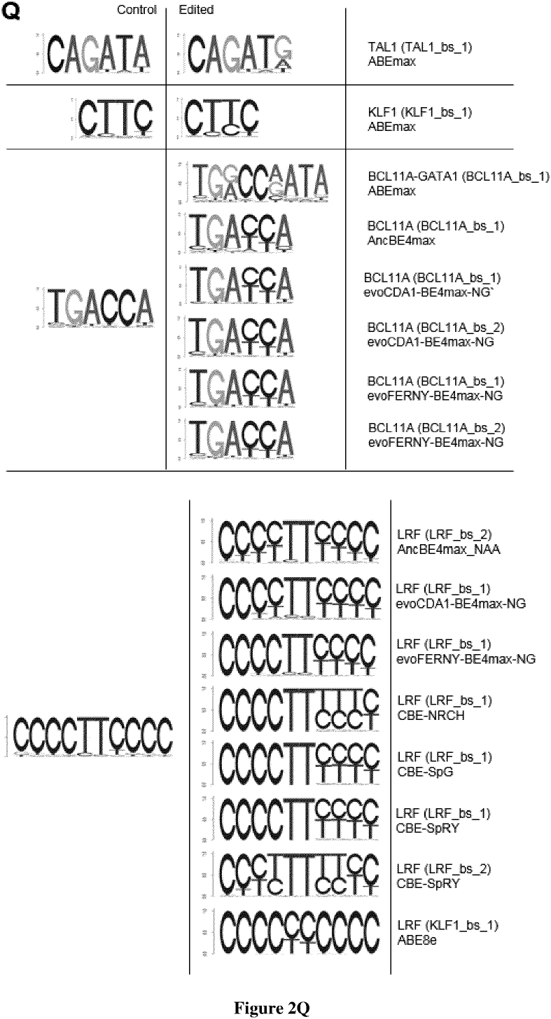

The -175 T>C HPFH mutation has been shown to recruit TAL1 transcription activator to the HBG promoters⁽⁶⁾, while the -198 T>C HPFH mutation recruits the KLF1 transcriptional activator⁽⁷⁾. We used gRNAs (TAL1_bs_1 and KLF1_bs_1) (FIG. 1) that can target bases in positions -175 and -198 in the HBG promoters. For these gRNAs, the target bases are in position 3 and 7, respectively. Transfection of the erythroleukemia cell line K562 with the ABEmax_GFP plasmid and the TAL1_bs_1 or KLF1_bs_1 gRNA plasmid, led to an A>G conversion (T>C in the opposite strand) with efficiencies of 61.0% and 30.3% respectively, in the bulk cell populations (FIGS. 2A, 2B and 2Q).

### Generation of TAL1 and KLF1 Binding Sites Leads to Γ-Globin De-Repression

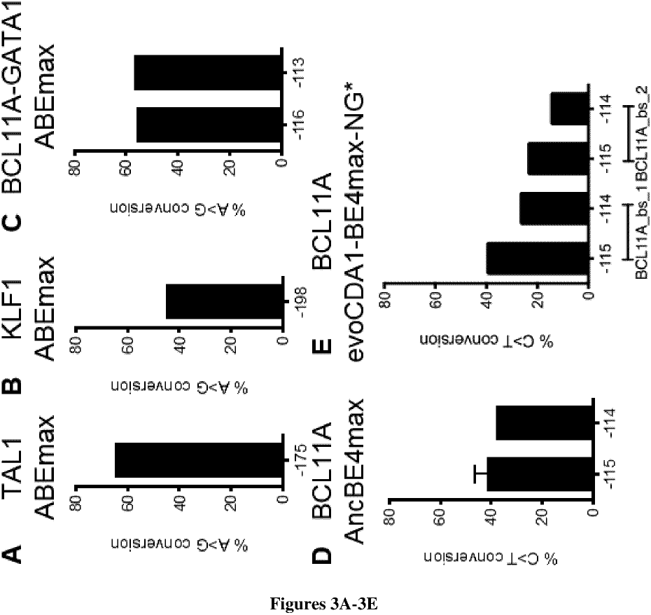

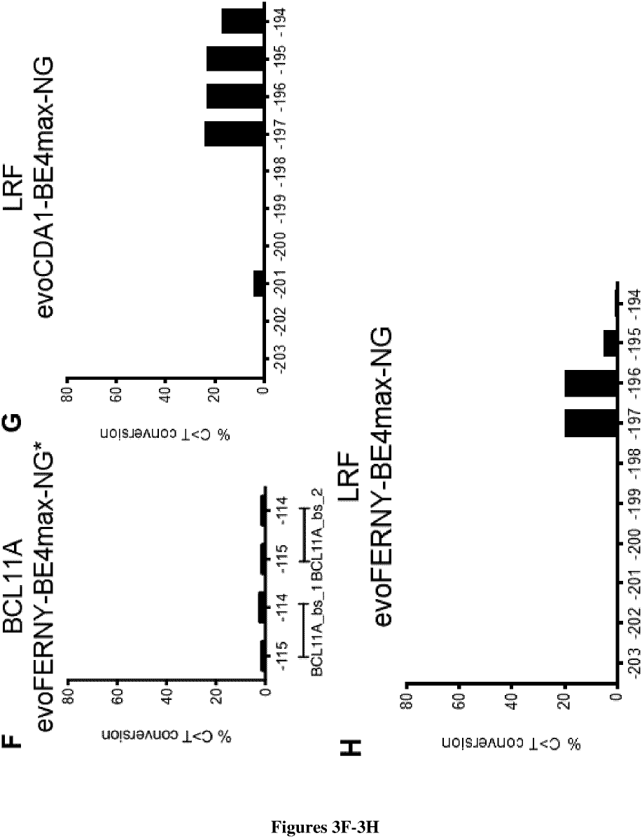

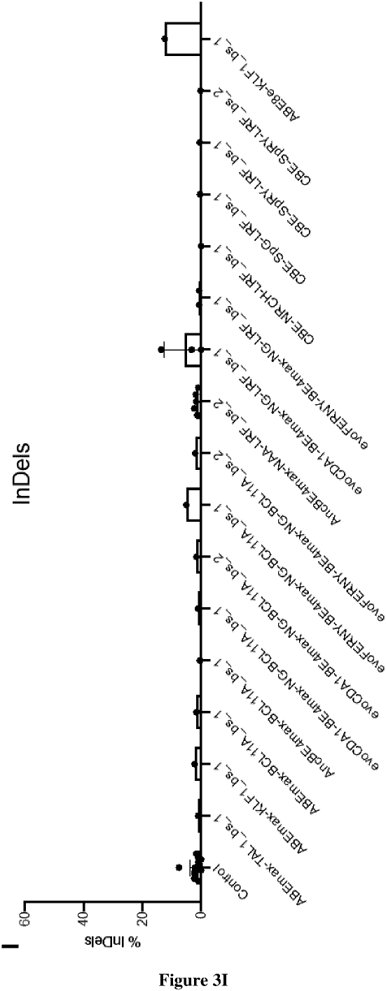

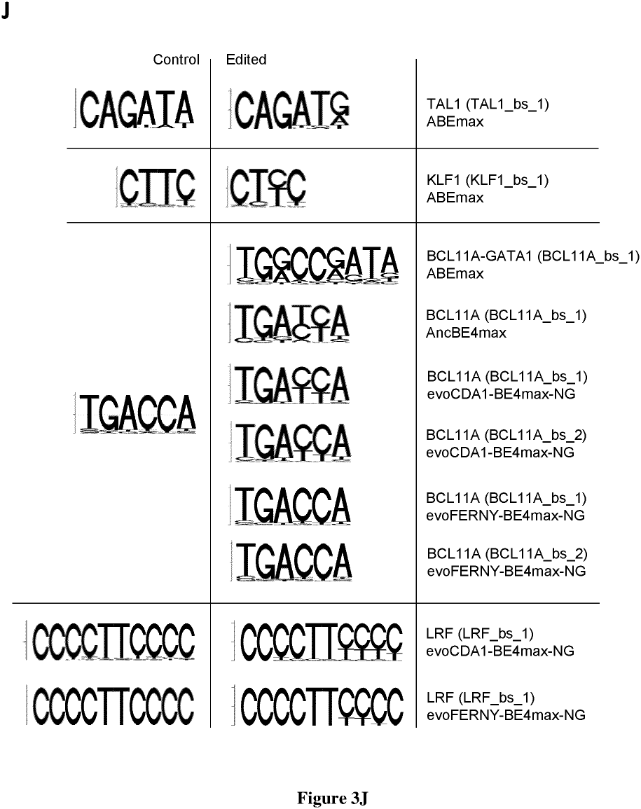

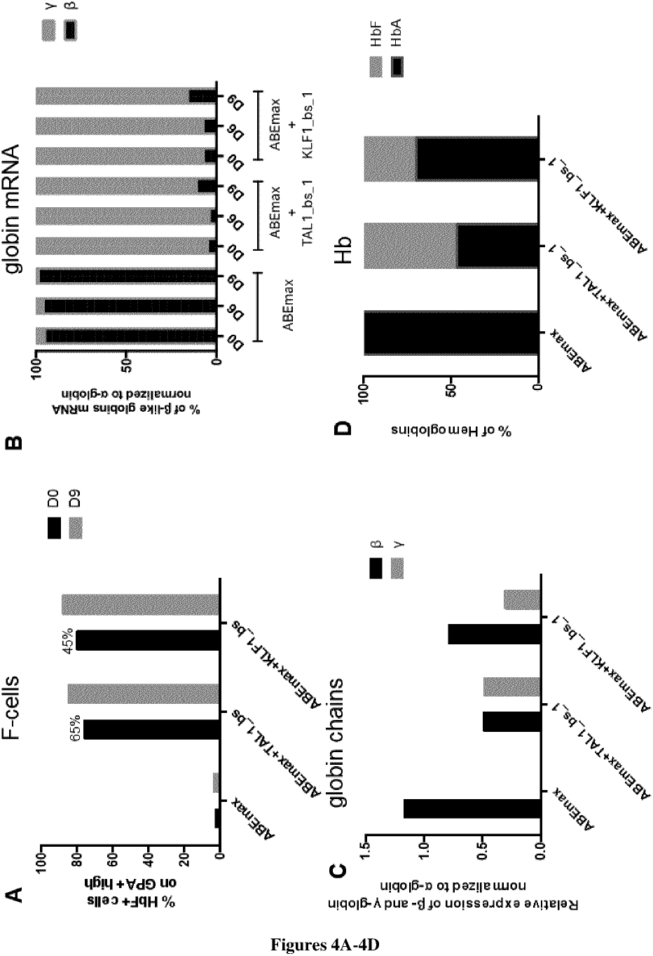

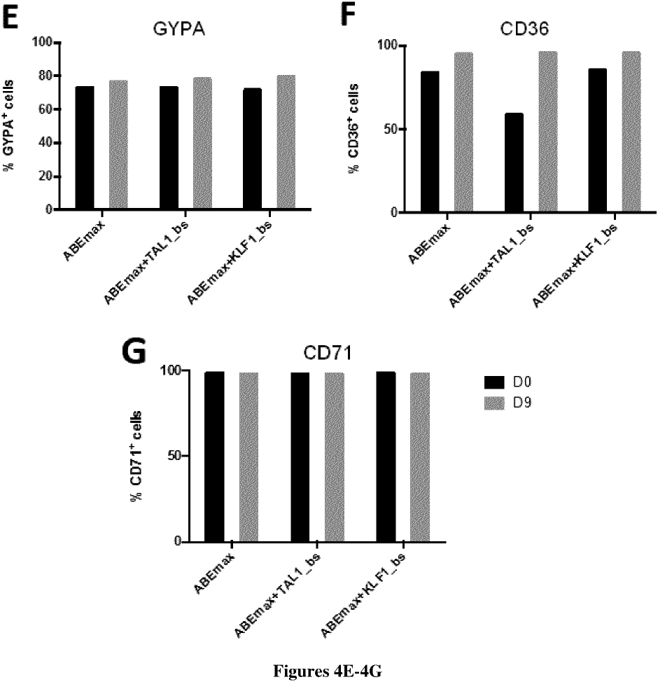

K562 cells express mainly γ-globin and for this reason they cannot be used as a model to measure γ-globin de-repression. Hence, we employed the HUDEP-2 adult erythroid progenitor cell line to evaluate γ-globin de-repression following the creation of the TAL1 and KLF1 activator binding sites. After plasmid transfection, with the ABEmax_GFP plasmid and either the TAL1_bs_1, or the KLF1_bs_1 gRNA plasmid, GFP⁺ HUDEP2 cells were sorted and expanded. The base editing efficiency was 65% and 45% at position -175 and -198, respectively (FIGS. 3A, 3B and 3J). Control and edited cells were differentiated in mature erythrocytes. Flow cytometry analysis of cells edited at -175 and -198 positions, revealed a high frequency of HbF-expressing cells (76.0% and 80.3% at day 0, and 85.0% and 88.1% at day 9 of differentiation), while in control populations transfected only with the ABEmax_GFP plasmid, the HbF-expressing cells were around 3.0% (FIG. 4A). Accordingly, we observed an increased production of γ-globin transcripts and a parallel decrease of the adult β-globin mRNAs (FIG. 4B). Reverse phase high-performance liquid chromatography (RP-HPLC) confirmed the significant increase in γ-globin with concomitant decrease of β-globin production (FIG. 4C). Generating the binding sites of TAL1 and KLF1 resulted in high HbF levels accounting for up to 53.5% and 30.0% respectively of the total Hb as determined by cation-exchange HPLC (CE-HPLC) (FIG. 4D). The base editing of the HBG½ promoters did not alter erythroid cell differentiation, as assessed by flow cytometry analysis of the erythroid markers GYPA, CD36 and CD71 (FIGS. 4E-4G).

### Base Editing in the -115 Region of the HBG Promoters Leads to the Disruption of the BCL11A Transcriptional Repressor Binding Site and the Simultaneous Generation of a Binding Site for GATA1 Transcriptional Activator

HPFH mutations on the -115 region of the HBG promoters cause elevated HbF expression by disrupting the binding site of the BCL11A transcriptional repressor (-114 C>T) or by creating a binding site for GATA1 transcriptional activator (-113 A>G). We designed gRNAs (BCL11A_bs_1 and BCL11A_bs_2) that can be used, either by ABE or CBE, in order to create these HPFH mutations (-114 C>T and -113 A>G) and additional HPFH-like mutations (-116 A>G and -115 C>T) (FIG. 1). The target bases fall into the canonical base editing window. In particular, the -116, -115, -114 and -113 bases are in positions 5-8 and 4-7 for the gRNAs BCL11A_bs_1 and BCL11A_bs_2 respectively. After transfection of the K562 cell line with the ABEmax_GFP and gRNA BCL11A_bs_1 plasmid, we succeeded to obtain the A>G conversions in position -116 and -113 (40.7% and 34.3% respectively) (FIGS. 2C and 2Q). Transfection of the same gRNA BCL11A_bs_1 plasmid with the AncBE4max_GFP plasmid, led to the C>T conversion in positions -115 and -114 (34.8% and 28.5% respectively) (FIGS. 2D and 2Q). In an effort to expand the enzyme options for disrupting the BCL11A transcriptional repressor binding site, we transfected the K562 cell line with the evoCDA1-BE4max-NG or the evoFERNY-BE4max-NG plasmid, two enzymes that recognize NG PAM, in combination with the two gRNAs targeting the BCL11A binding site (BCL11A_bs_1 and BCL11A_bs_2). All of the 4 different combinations led to the same C>T conversions (-115 C>T and -114 C>T) with efficiencies ranging from 18 to 40.5% (evoCDA1-BE4max-NG -BCL11A_bs_1; 40.5% -115 C>T and 31.5% -114 C>T, evoCDA1-BE4max-NG -BCL11A_bs_2; 30.0% -115 C>T and 22.0% -114 C>T, evoFERNY-BE4max-NG -BCL11A_bs_1; 22.0% -115 C>T and 21.5% -114 C>T and evoFERNY-BE4max-NG -BCL11A_bs_2; 18.0% -115 C>T and 19.0% -114 C>T) (FIGS. 2E, 2F and 2Q).

### Non-NGG Base Editors Can Disrupt the LRF Transcriptional Repressor Binding Site By Editing Multiple Bases and Creating HPFH and HPFH-Like Mutations

The -200 region of the HBG promoters contains different HPFH mutations associated with high expression of γ-globin in adult life. The majority of these mutations de-repress the HBG genes by reducing the binding capacity of the LRF transcriptional repressor via the disruption of its binding site. In the LRF binding site, there are 8 cytosines and theoretically all of them can be targeted by base editing in order to be converted in thymines. Consequently, it is possible to create multiple HPFH mutations and additional mutations that could induce an HPFH-like phenotype by impairing the LRF binding capacity (FIG. 1). The absence of the canonical SpyCas9 NGG PAM close to the LRF binding site prompted us to generate base editing enzymes containing non-NGG Cas9 variants that allowed the editing of this site. This variant of Cas9 recognizes an NAA PAM, which is ideal for targeting the LRF binding site, as it allows the designing of a gRNA (LRF_bs_2) that places the target bases - 8 cytosines - in position 2-11. After the PAM-interacting domain exchange, the resulting enzyme (called AncBE4max_NAA) was transfected as plasmid in the K562 cell line in combination with the LRF_bs_2 gRNA plasmid. We were able to modify 7 out of the 8 cytosines of the LRF binding site with efficiencies of up to 37.0% (8.7% -202 C>T; 20.3% -201 C>T; 37.0% -200 C>T; 30.7% -197 C>T; 27.7% -196 C>T; 16.3% -195 C>T; 5.7% -194 C>T) (FIGS. 2G and 2Q). One more gRNA (LRF _bs_1) was designed so as to target the LRF binding site using the evoCDA1-BE4max-NG or the evoFERNY-BE4max-NG enzyme (FIG. 1). With these combinations we can target 6 out of 8 cytosines of the motif and these 6 cytosines are in position 1-8. Transfection of the K562 cell line with the LRF_bs_1 gRNA plasmid and either the evoCDA1-BE4max-NG or the evoFERNY-BE4max-NG enzyme plasmid revealed efficiencies that ranged from 8.0 to 28.5% for the evoCDA1-BE4max-NG enzyme (8.0% -201 C>T; 22.5% -200 C>T; 28.0% -197 C>T; 27.5% -196 C>T; 28.5% -195 C>T; 21.0% -194 C>T) and from 15.0 to 32.5% for the evoFERNY-BE4max-NG enzyme (28.0% -197 C>T; 32.5% -196 C>T; 25.5% -195 C>T; 15.0% -194 C>T) (FIGS. 2H, 2I and 2Q). The same gRNAs were tested with more efficient non-NGG base editors, such as CBE-NRCH, CBE-SpG and CBE-SpRY. When combined with LRF_bs_1 gRNA, CBE-NRCH, CBE-SpG and CBE-SpRY led to C>T efficiencies up to 50.3%, 43.7% and 46.3% respectively, upon plasmid transfection in K562 cells (FIGS. 2J-L and 2Q). Given the PAMless nature of CBE-SpRY, this base editor was combined with LRF_bs_2 gRNA and outperformed all the above-mentioned enzymes hitting efficiencies of 58.0%. Importantly thanks to its wide editing window, CBE-SpRY converted all the cytosines of the -200 region (FIGS. 2M and 2Q). Finally, an ABE8e enzyme plasmid was co-transfected in K562 cells with the KLF1_bs_1 gRNA and led to A>G modifications of both the -198 A:T bp and the -199 A:T bp with efficiencies of up to 72.7%, resulting in LRF binding site disruption (FIGS. 2N and 2Q).

### Disruption of the BCL11A and LRF Transcriptional Repressor Binding Sites by Base Editing Leads to HbF Reactivation

The previously reported base editing approaches for targeting the -115 and -200 regions of the HBG promoters, by creating HPFH and HPFH-like mutations, were tested in the HUDEP-2 cell line in order to evaluate the γ-globin de-repression, after disrupting the BCL11A and LRF transcriptional repressor binding sites, and/or after creating a binding site for GATA1 transcriptional activator. For both regions, plasmids expressing 4 different enzymes (ABEmax_GFP, AncBE4max_GFP, evoCDA1-BE4max-NG and evoFERNY-BE4max-NG) were individually transfected in combination with single gRNA-expressing plasmids (BCL11A_bs_1, BCL11A_bs2, LRF_bs_1 and LRF_bs_2) in HUDEP-2 cells. For plasmids that do not express GFP (evoCDA1-BE4max-NG and evoFERNY-BE4max-NG), a small amount of a GFPmax-expressing plasmid was co-transfected. After transfection, GFP⁺ cells were FACS-sorted, expanded in culture and differentiated in erythrocytes.

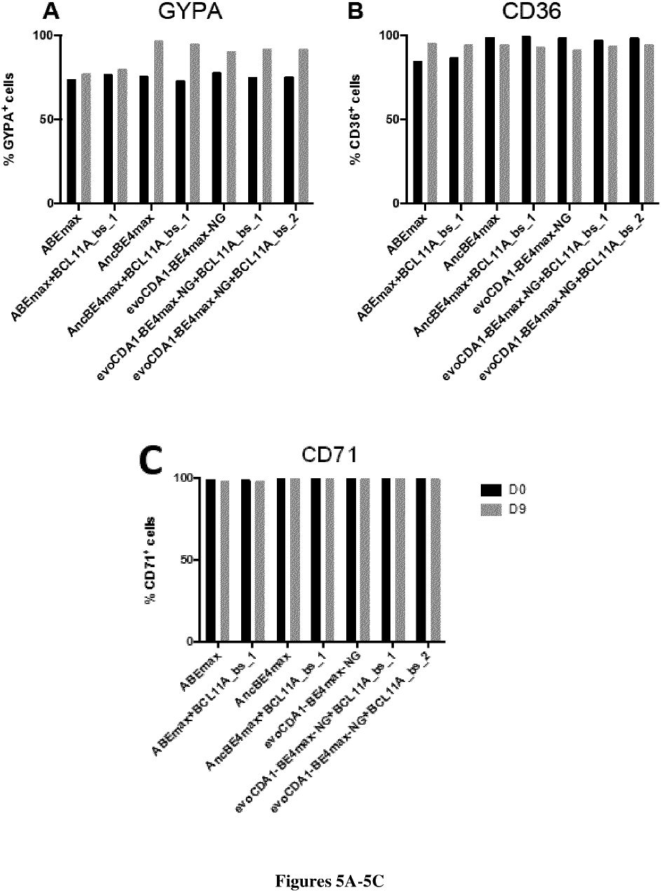

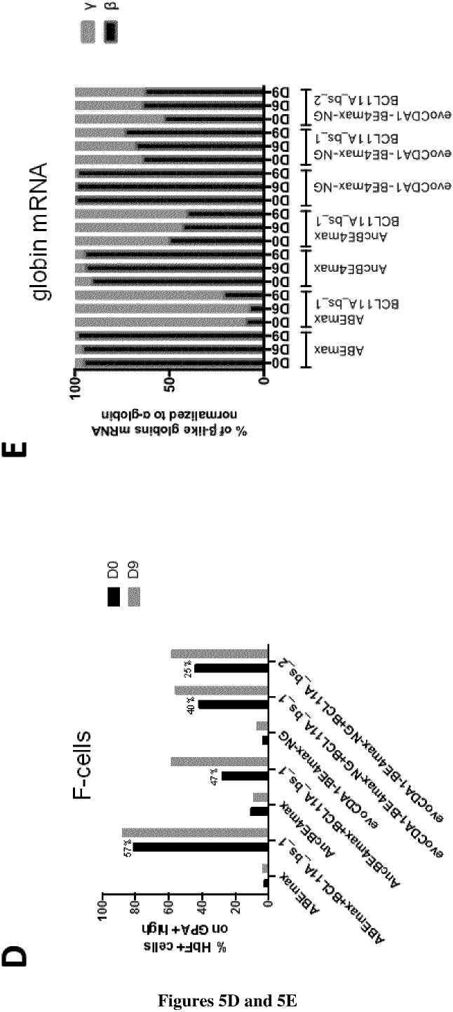

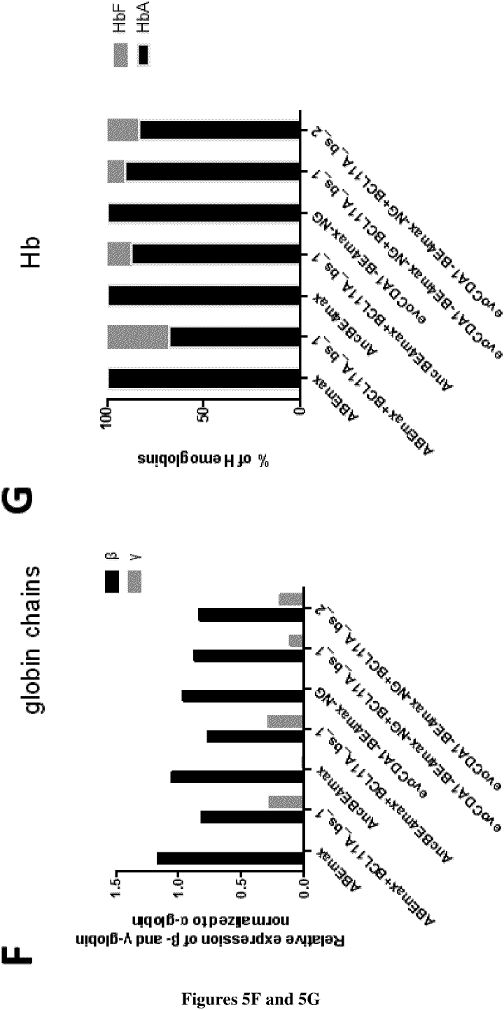

Editing the BCL11A binding site with the ABEmax_GFP enzyme and the BCL11A_bs_1 gRNA, led to -116 A>G and -113 A>G conversions, with a frequency of 56.0% and 57.0%, respectively, disrupting the BCL11A binding site and simultaneously creating a GATA1 binding site (FIGS. 3C and 3J). Using the AncBE4max_GFP enzyme combined with the BCL11A_bs_1 gRNA, we succeeded in generating HPFH (38.0% -114 C>T) and HPFH-like (47.0% -115 C>T) mutations (FIGS. 3D and 3J). Base editing by the non-NGG PAM enzymes, (evoCDA1-BE4max-NG and evoFERNY-BE4max-NG) in combination with either the BCL11A_bs_1 or the BCL11A_bs_2 gRNA, was effective only using evoCDA1-BE4max-NG that led to -115 C>T and -114 C>T conversions (40.0% and 27.0%, respectively) with the BCL11A_bs_1 gRNA and -115 C>T and -114 C>T conversions (24.0% and 15.0%, respectively) with the BCL11A_bs_2 gRNA (FIGS. 3E, 3F and 3J). Base editing of the BCL11A binding site with the above-mentioned enzymes did not affect the erythroid differentiation, as assessed by flow cytometry analysis of the erythroid markers GYPA, CD36 and CD71 (FIGS. 5A-5C). Flow cytometry analysis showed an increased frequency of HbF-expressing cells (up to 87.8%) (FIG. 5D). RTqPCR analysis, in accordance with the flow cytometry data, revealed elevated production of γ-globin transcripts with a concomitant decrease of β-globin transcripts production (FIG. 5E). RP-HPLC and CE-HPLC analysis confirmed these data, with HbF representing up to 31.8% of the total Hb (FIGS. 5F-5G).

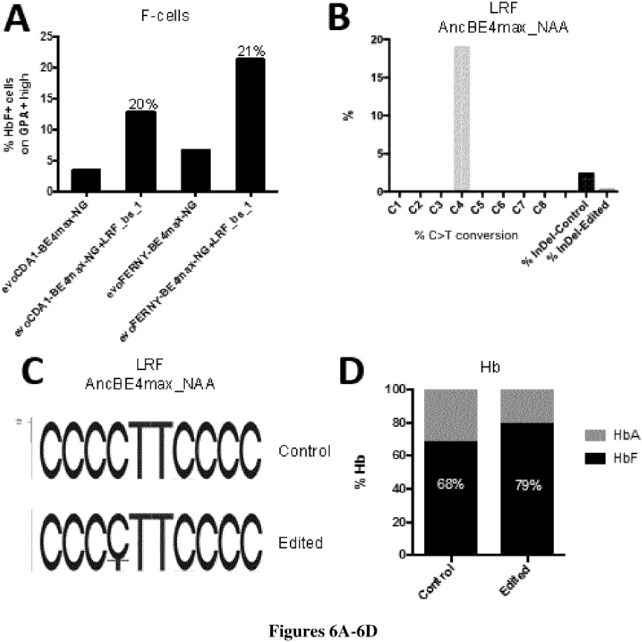

The use of evoCDA1-BE4max-NG and evoFERNY-BE4max-NG enzymes, combined with the LRF_bs_1 gRNA, to target the LRF repressor binding site, led to base editing efficiencies of up to 24.0% for evoCDA1-BE4max-NG enzyme (4.0% -201 C>T; 24.0% -197 C>T; 23.0% -196 C>T; 23.0% -195 C>T; 17.0% -194 C>T) and up to 20.0% for evoFERNY-BE4max-NG enzyme (20.0% -197 C>T; 20.0% -196 C>T; 5.0% -195 C>T; 1.0% -194 C>T) (FIGS. 3G, 3H and 3J), with a concomitant increase of the HbF-expressing cells to 12.9% (3.5% in the non-edited control cells) and 21.4% (6.7% in the non-edited control cells) respectively (FIG. 6A). We then used primary cord blood-derived CD34⁺ hematopoietic stem/progenitor cells (HSPCs). CD34+ cells were transfected with the AncBE4max_NAA, the LRF_bs_1 gRNA and the GFPmax plasmids and sorted for GFP expression. Cells were either maintained in liquid culture or plated in a semi-solid medium allowing the growth and differentiation of erythroid and granulocyte-monocyte progenitors (colony forming unit assay). 6 days post-transfection, we obtained an 19.0% efficiency of -200 C>T conversion in the liquid culture (FIGS. 6B-6C). 14 days post-transfection cation-exchange HPLC analysis was performed to detect hemoglobin tetramers in the burst-forming unit-erythroid (BFU-E) colonies derived from erythroid progenitors (FIG. 6D). This analysis revealed an increase in HbF level of 11.02% (68.22% in the control samples and 79.24% in the edited sample) (FIG. 6D). Altogether these data show that the novel AncBE4max_NAA base editing enzyme can target the LRF transcriptional repressor binding site in primary HSPCs and increase the HbF expression in their erythroid progeny.

### InDels and Large Deletions Were Barely Detectable in Base-Edited Cells

One of the safety issues that emerges with the usage of CRISPR/Cas9 nuclease is the creation of Insertions and Deletions (InDels) in the genome after the generation of double strand breaks. Though, with the base editing system we can overcome this issue, as base editors contain an inactivated Cas9 nuclease. We wanted to verify that we do not create double strand breaks in the genome with the base editing enzymes that we employed. For this reason, we amplified the target regions by PCR, and performed Sanger sequencing followed by TIDE analysis⁽²⁸⁾ for base-edited and control K562 samples (FIGS. 2O, 3I and FIG. 6B). For almost all of the samples, we detected no InDels, except for cells transfected with ABE8e and KLF1_bs_1 gRNA plasmids, and evoCDA1-BE4max-NG and LRF_bs_2 gRNA plasmid, showing an average of 18.4% and 22.0% of InDels respectively (FIG. 2O).

Another issue that arises when editing the β-globin locus with the Cas9 nuclease, is the simultaneous cleavage of the identical HBG½ promoters resulting in the deletion of the intervening 4.9-kb genomic region and loss of the HBG2 gene. Therefore, we tested if the 4.9-kb deletion was present in base-edited K562 samples. In accordance with the InDel profile of base edited samples (FIG. 2O), we observed a low frequency of the 4.9-kb deletion only in ABEmax-, ABE8e- and evoFERNY-BE4max-NG-treated samples (4.9%, 4.9% and 3.2%, respectively; FIG. 2P). As a positive control for the 4.9-kb deletion, we used DNA extracted from K562 cells edited with the canonical Cas9 nuclease (FIG. 2P).

### Enzymes With Low RNA Off-Target Activity Can Be Used to Target the HBG Repressors and Activators Binding Sites

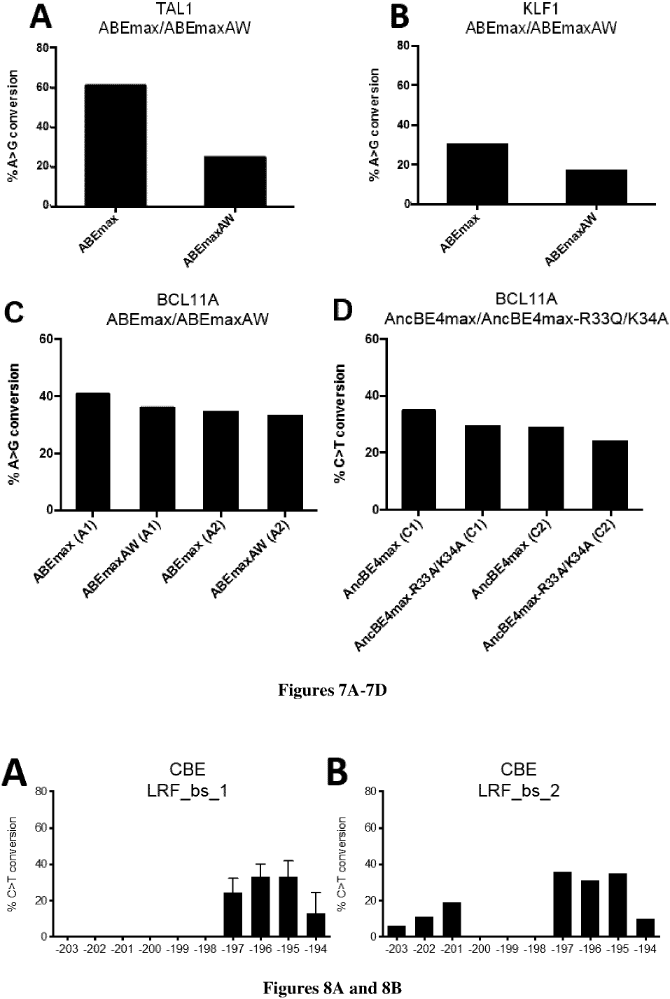

Base editing enzymes can cause off-target editing of the cellular RNA that mostly is gRNA-independent. Mutations in the deaminase of the base editing enzyme can minimize RNA off-target editing. For the adenine base editors, these mutations are the E59A in the TadA domain and the V106W in the TadA* domain and the enzyme that carries these mutations is called ABEmaxAW⁽²⁹⁾. The mutations for the cytosine base editors are the R33A and the K34A in the APOBEC1 domain⁽³⁰⁾. By inserting these mutations in the AncBE4max enzyme, we created the AncBE4max_R33A/K34A base editing enzyme. Our purpose was to verify if these enzymes, with low RNA off-target editing, could be used to create the TAL1 and KLF1 activator binding sites or disrupt the BCL11A repressor binding site. Transfection of K562 cells with the ABEmaxAW plasmid and the TAL1_bs_1 or the KLF1_bs_1 gRNA plasmid led to -175 T>C (A>G to the opposite strand) and T>C (A>G to the opposite strand) conversion, with a frequency of 25.0% and 17.0%, respectively (FIGS. 7A and 7B). Targeting the BCL11A binding site with the ABEmaxAW in combination with the BCL11A_bs_1 gRNA plasmid, caused 36.0% -116 A>G and 33.0% -113 A>G conversions (FIG. 7C), while targeting this site with the same gRNA and the AncBE4max_R33A/K34A enzyme led to -115 C>T and -114 C>T conversions, with a frequency of 29.0% and 24.0%, respectively (FIG. 7D). Altogether, these data demonstrate that these safer versions of base editing enzymes can be used to efficiently target transcriptional activator or repressor binding sites in the HBG promoters.

### Disruption of the LRF Repressor Binding Site in the HBG Promoters by CBEs in SCD HSPCs Leads to HbF Reactivation and Rescues the Sickling Phenotype

To prove the efficacy of our base editing approaches as therapeutic strategies for the treatment of SCD, we transfected base editor- and gRNA-expressing plasmids in primary human adult non-mobilized SCD HSPCs. In particular, plasmids expressing CBE enzymes (CBE-NRCH, CBE-SpG-GFP, CBE-SpRY-GFP) were individually transfected in combination with single gRNA-expressing plasmids (LRF_bs_1, LRF_bs_2). To enrich for edited cells, either we used plasmids that express base editor-GFP fusions (CBE-SpG-GFP, CBE-SpRY-GFP) or we co-transfected base editor- (CBE-NRCH) and GFPmax-expressing plasmids. GFP^{high} cells were FACS-sorted differentiated toward the erythroid lineage using a 3-phase protocol.

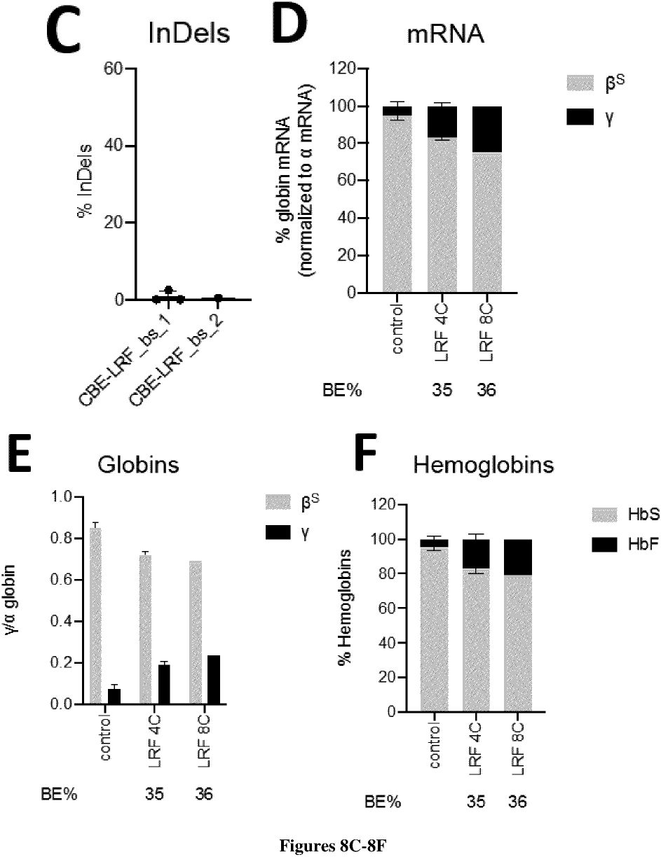

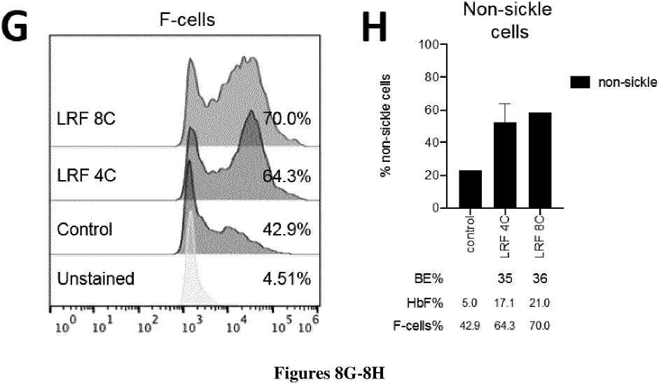

Base editing efficiency was measured in erythroblasts at the end of the first phase of erythroid differentiation (Day 6). Samples treated with CBEs and LRF_bs_1 gRNA, converting 4C in the LRF binding site (LRF 4C), showed editing efficiencies of ~22.4% (26.8%, 23.8% and 16.5% with CBE-NRCH, CBE-SpG and CBE-SpRY respectively) (FIG. 8A). All the cytosines of the LRF binding site were converted into T in CBE-SpRY- and LRF_bs_2-treated samples (LRF 8C) with efficiencies of up to 25.5% (FIG. 8B). TIDE analysis in base-edited samples confirmed the absence of InDels (FIG. 8C).

We then differentiated bulk populations of edited erythroblasts into mature RBCs to evaluate HbF expression and the recovery of the sickling cell phenotype. The erythroid differentiation was similar between control and CBE-treated samples, as measured by flow cytometry analysis of late (CD36, CD71 and α4-Integrin) and early (GYPA and BAND3) erythroid markers along the differentiation (data not shown). The enucleation rate was similar between groups at different time points throughout the differentiation and at the end of the last phase reached more than 90% in all samples (data not shown). LRF 4C and LRF 8C CBE-treated samples showed fetal hemoglobin reactivation at both at mRNA and protein level, as measured by RT-qPCR, RP-HPLC (FIGS. 8D and 8E) and CE-HPLC (22.3% and 9.1% HbF in edited samples and control samples, respectively; FIG. 8F). Flow cytometry analysis revealed a high frequency of HbF-expressing RBCs (42.9%, 64.3% and 70.0% in control, LRF 4C and LRF 8C samples, respectively; FIG. 8G). To evaluate the effect of HbF reactivation on the sickling phenotype, we incubated mature RBCs under hypoxic conditions inducing HbS polymerization. Interestingly, editing of either 4 or 8 C of the LRF binding site ameliorated the sickling phenotype (23.2%, 52.5%- and 58.4% of non-sickle cells in control, LRF 4C and LRF 8C samples respectively) (FIG. 8H). Overall, these data demonstrate that base editing of the HBG½ promoters by CBEs can lead to HbF reactivation and rescue the sickling phenotype of RBCs differentiated from SCD patient HSPCs.

### Disruption of the LRF Repressor Binding Site or Creation of the KLF1 Activator in the HBG Promoters by ABEs in SCD HSPCs Leads to HbF Reactivation and Rescues the Sickling Phenotype

ABEs can be also used in order to disrupt the LRF transcriptional repressor binding site or to create the KLF1 transcriptional activator binding site. We performed the same set of experiments described in the previous paragraph for CBEs in primary human adult non-mobilized SCD HSPCs to demonstrate ABEs′ therapeutical potential. More specifically, plasmids expressing ABEmax-GFP or ABE8e were individually transfected in combination with single gRNA-expressing plasmids (KLF1_bs_1 gRNA or AAVS1 gRNA targeting the unrelated AAVS1 locus; Weber et al., Sc. Advances, 2020). To enrich for edited cells, we used a plasmid expressing the ABEmax-GFP fusion protein or we co-transfected ABE8e- and GFPmax-expressing plasmids. After transfection, GFP^{medium} and GFP^{high} cells were FACS-sorted to obtain cell populations with a variety of editing efficiencies. Sorted cells were fully differentiated into mature RBCs using a 3-phase protocol.

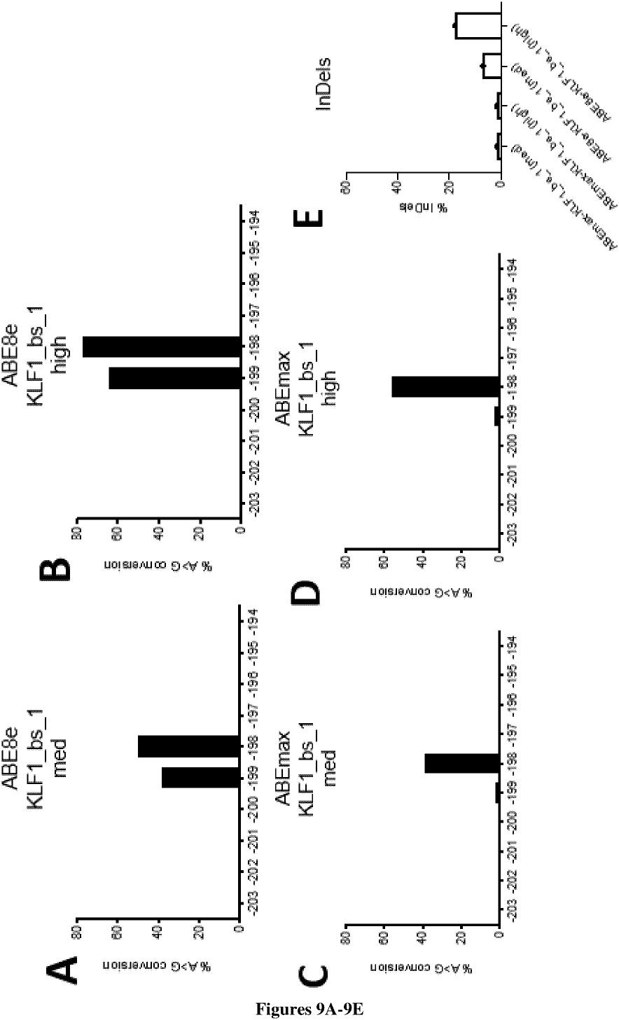

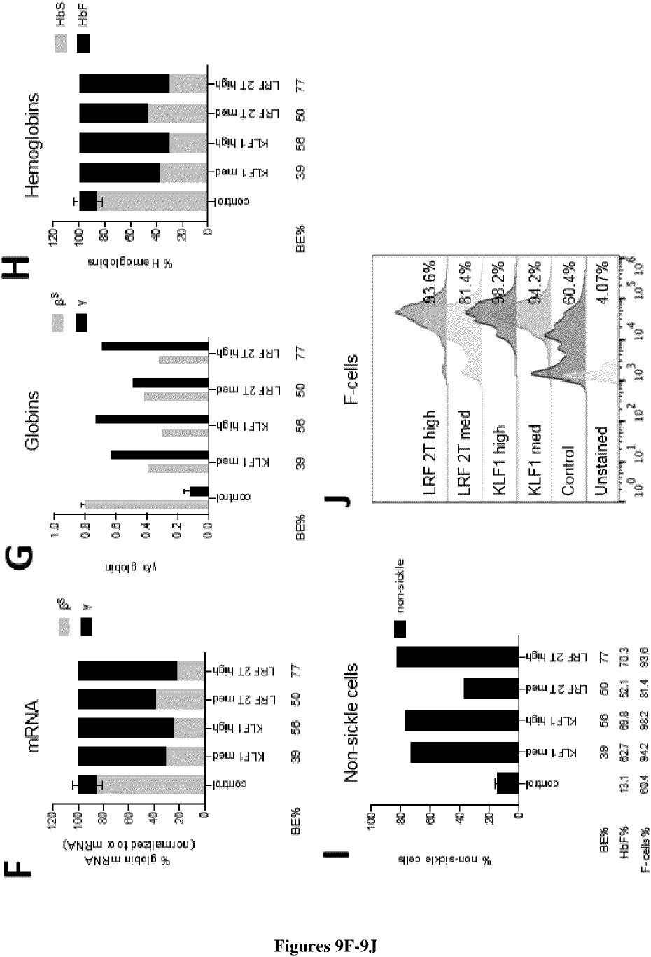

Base editing efficiency was measured in erythroblasts at the end of the first phase of erythroid differentiation (Day 6). ABEmax (generating a KLF1 binding site, KLF1)- and ABE8e (converting the 2 T of the LRF binding site; LRF 2T) treated samples showed efficiencies that ranged from 41.0% to 52.3%, and 56.5% to 76.0% in the GFP^{medium} and GFP^{high} bulk populations, respectively (FIGS. 9A-9D). TIDE analysis in the base edited samples confirmed absence of InDels for ABEmax-treated cells (FIG. 9E), while a moderate InDel frequency of 7.9% and 14.8% was detected in GFP^{medium} and GFP^{high} ABE8e-treated samples, respectively (FIG. 9E).

Differentiation of bulk populations of edited erythroblasts into mature RBCs was performed to evaluate HbF expression and recovery of the sickling cell phenotype. The erythroid differentiation was similar between control and ABE-treated samples, as measured by flow cytometry analysis of late (CD36, CD71 and α4-Integrin) and early (GYPA and BAND3) erythroid markers along the differentiation (data not shown). The enucleation rate was similar between groups at different time points throughout the differentiation and at the end of the last phase reached more than 90% in all samples (data not shown). ABE-treated samples, bearing either the KLF1 binding site or the LRF 2T profile, expressed high HbF levels (66.3% and 62.6% respectively), as measured by CE-HPLC (FIG. 9H). These results were confirmed by RT-qPCR and RP-HPLC at mRNA and single globin chain levels (FIGS. 9F and 9G). Flow cytometry analysis revealed a high frequency of HbF-expressing RBCs (60.4%, ≥94.2% and ≥81.4% in control, ABEmax- and ABE8e-treated samples, respectively) (FIG. 9J). A sickling assay was performed in control and edited samples. High frequencies of corrected cells were observed for ABEmax- and ABE8e-treated samples (14.7%, 75.7% and 60.0% of non-sickle cells in control, ABEmax- and ABE8e-treated samples, respectively) (FIG. 9I). Overall, this study shows that either disrupting the LRF repressor binding site or creating the KLF1 activator binding site in the -200 region of the HBG½ promoters using ABEs leads to fetal hemoglobin reactivation and rescues the sickling phenotype in RBCs differentiated from SCD patient HSPCs.

### Efficient RNA-Mediated Editing of the HBG½ Promoters in K562 Cells and SCD HSPCs

To establish a clinically relevant method to deliver the base editing system in primary HSPCs and achieve a high editing efficiency coupled with minimal toxicity, we optimized a protocol based on transfection of mRNA encoding base editors and synthetic modified gRNAs. First, we optimised the plasmids encoding CBE-SpRY and ABE-SpRY for in vitro transcription and mRNA production. In particular, we inserted two copies of the 3′ untranslated region (UTR) of the HBB gene (which has been shown to increase the half-life of mRNA and improve protein levels³¹⁻³³) and a poly-A sequence after the 3′ UTR to further stabilize the mRNA³⁴ in CBE-SpRY and ABE-SpRY constructs.

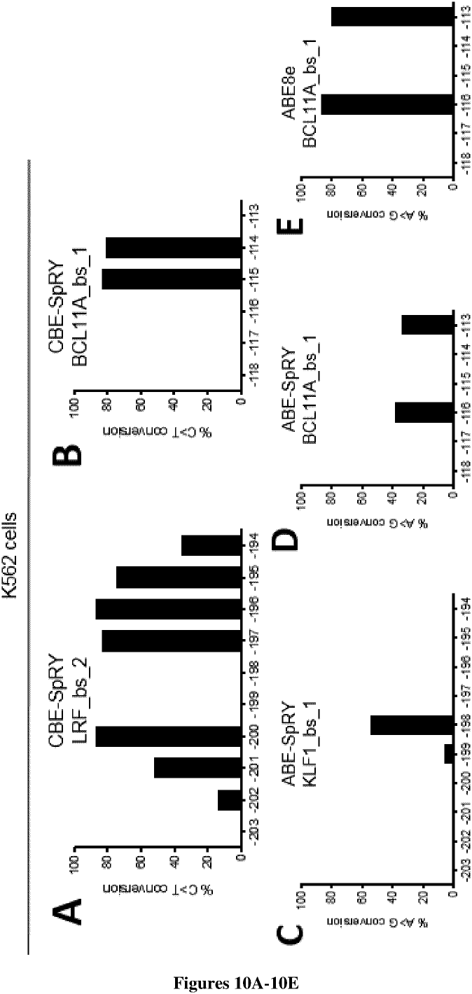

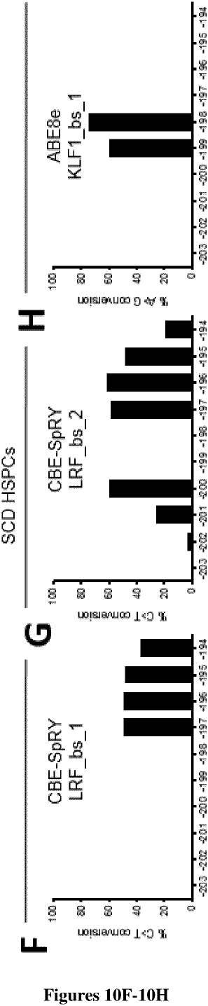

Next, we performed in vitro mRNA transcription using CBE-SpRY-OPT, ABE-SpRY-OPT and ABE8e plasmids. In K562 cells, transfection of CBE-SpRY, ABE-SpRY and ABE8e mRNAs together with LRF_bs_2, KLF1_bs_1 or BCL11A_bs_1 synthetic modified gRNAs led to high base editing efficiencies, demonstrating that we generated fully functional mRNAs. In particular, transfection of CBE-SpRY mRNA in combination with LRF_bs_2 or BCL11A_bs_1 gRNA resulted in 87.0% and 83.0% of C>T conversion, respectively (FIGS. 10A and 10B). Similarly, transfection of ABE-SpRY mRNA and KLF1_bs_ or BCL11A_bs_1 gRNA resulted in 55.0% and 39.0% of A>G conversion, respectively. Finally, co-transfection of ABE8e mRNA and BCL11A_bs_1 gRNA led to 88.0% of base editing efficiency (FIGS. 10C-10E).

CBE-SpRY and ABE8e mRNAs were transfected also in SCD HSPCs in combination with chemically modified single gRNAs. CBE-SpRY mRNA coupled with LRF_bs_1 or LRF_bs_2 gRNA led to 51.0% and 61.0% of C>T conversion respectively, while ABE8e mRNA coupled with KLF1_bs_1 gRNA led to 75.0% A>G conversion (FIGS. 10F-10H). These results demonstrate that RNA-mediated delivery of base editors allows efficient targeting of the HBG½ promoters in SCD HSPCs.

### REFERENCES

Throughout this application, various references describe the state of the art to which this invention pertains. The disclosures of these references are hereby incorporated by reference into the present disclosure.

| | |
|---|---|
| 1 | Taher, Weatherall, and Cappellini, ‘Thalassaemia’. |
| 2 | Kato et al., ‘Sickle Cell Disease’. |
| 3 | Chandrakasan and Malik, ‘Gene Therapy for Hemoglobinopathies’. |
| 4 | Cavazzana, Antoniani, and Miccio, ‘Gene Therapy for β-Hemoglobinopathies’. |
| 5 | Forget, ‘Molecular Basis of Hereditary Persistence of Fetal Hemoglobin’. |
| 6 | Wienert et al., ‘Editing the Genome to Introduce a Beneficial Naturally Occurring Mutation Associated with Increased Fetal Globin’. |
| 7 | Wienert et al., ‘KLF1 Drives the Expression of Fetal Hemoglobin in British HPFH’. |
| 8 | Martyn et al., ‘A Natural Regulatory Mutation in the Proximal Promoter Elevates Fetal Globin Expression by Creating a de Novo GATA1 Site’, 1. |
| 9 | Martyn, Quinlan, and Crossley, ‘The Regulation of Human Globin Promoters by CCAAT Box Elements and the Recruitment of NF-Y′. |
| 10 | Antoniani et al., ‘Induction of Fetal Hemoglobin Synthesis by CRISPR/Cas9-Mediated Editing of the Human β-Globin Locus’. |
| 11 | Weber, Frati et al., ‘Editing a γ-Globin Repressor Binding Site Restores Fetal Hemoglobin Synthesis and Corrects the Sickle Cell Disease Phenotype’. |
| 12 | Truong et al., ‘Microhomology-Mediated End Joining and Homologous Recombination Share the Initial End Resection Step to Repair DNA Double-Strand Breaks in Mammalian Cells’. |
| 13 | Milyavsky et al., ‘A Distinctive DNA Damage Response in Human Hematopoietic Stem Cells Reveals an Apoptosis-Independent Role for P53 in Self-Renewal’. |
| 14 | Cromer et al., ‘Global Transcriptional Response to CRISPR/Cas9-AAV6-Based Genome Editing in CD34+ Hematopoietic Stem and Progenitor Cells’. |
| 15 | Haapaniemi et al., ‘CRISPR-Cas9 Genome Editing Induces a P53-Mediated DNA Damage Response’. |
| 16 | Kosicki, Tomberg, and Bradley, ‘Repair of Double-Strand Breaks Induced by CRISPR-Cas9 Leads to Large Deletions and Complex Rearrangements’. |
| 17 | Gaudelli et al., ‘Programmable Base Editing of A·T to G·C in Genomic DNA without DNA Cleavage’. |
| 18 | Rees and Liu, ‘Base editing: precision chemistry on the genome and transcriptome of living cells’. |
| 19 | Yeh et al., ‘In Vivo Base Editing of Post-Mitotic Sensory Cells’. |
| 20 | Masuda et al., ‘Transcription Factors LRF and BCL11A Independently Repress Expression of Fetal Hemoglobin’. |
| 21 | Koblan et al., ‘Improving Cytidine and Adenine Base Editors by Expression Optimization and Ancestral Reconstruction’. |
| 22 | Li et al., ‘Reactivation of γ-Globin in Adult β-YAC Mice after Ex Vivo and in Vivo Hematopoietic Stem Cell Genome Editing’. |
| 23 | Behera et al., ‘Exploiting Genetic Variation to Uncover Rules of Transcription Factor Binding and Chromatin Accessibility’. |
| 24 | Brinkman et al., ‘Easy Quantitative Assessment of Genome Editing by Sequence Trace Decomposition’ . |
| 25 | Kurita et al., ‘Establishment of Immortalized Human Erythroid Progenitor Cell Lines Able to Produce Enucleated Red Blood Cells’. |
| 26 | Nettling et al., ‘DiffLogo: a comparative visualization of sequence motifs’. |
| 27 | Kluesner et al., ‘EditR: A Method to Quantify Base Editing from Sanger Sequencing’. |
| 28 | Brinkman et al., ‘Easy Quantitative Assessment of Genome Editing by Sequence Trace Decomposition’. |
| 29 | Rees et al., ‘Analysis and minimization of cellular RNA editing by DNA adenine base editors’. |
| 30 | Grünewald et al., ‘Transcriptome-wide off-target RNA editing induced by CRISPR-guided DNA base editors’ . |
| 31 | Karikó, K., Kuo, A. & Barnathan, E. Overexpression of urokinase receptor in mammalian cells following administration of the in vitro transcribed encoding mRNA. Gene Ther. 6, 1092-1100 (1999). |
| 32 | Ross, J. & Sullivan, T. D. Half-lives of beta and gamma globin messenger RNAs and of protein synthetic capacity in cultured human reticulocytes. Blood 66, 1149-1154 (1985). |
| 33 | Holtkamp, S. et al. Modification of antigen-encoding RNA increases stability, translational efficacy, and T-cell stimulatory capacity of dendritic cells. Blood 108, 4009-4017 (2006). |
| 34 | Gallie, D. R. The cap and poly(A) tail function synergistically to regulate mRNA translational efficiency. Genes Dev. 5, 2108-2116 (1991). |

1. A method for increasing fetal hemoglobin content in a eukaryotic cell comprising contacting the eukaryotic cell with a gene editing platform comprising (a) at least one base-editing enzyme and (b) at least one guide RNA molecule for guiding the base-editing enzyme to at least one target sequence in the an HBG1 or HBG2 promoter, thereby editing said promoter and subsequently increasing the expression of gamma globin in said eukaryotic cell.

2. The method of claim 1 wherein the gene editing platform introduces the a -198T>C mutation in the HBG1 or HBG2 promoter so that the KFL1 activator binds to the HBG1 or HBG2 promoter.

3. The method of claim 1 wherein the gene editing platform introduces the a -175T>C mutation in the HBG1 or HBG2 promoter so that the TAL1 activator binds to the HBG1 or HBG2 promoter.

4. The method of claim 1 wherein the gene editing platform introduces a -113A>G mutation in the HBG1 or HBG2 promoter thereby permitting binding of a GATA1 activator to the HBG1 or HBG2 promoter.

5. The method of claim 1 wherein the gene editing platform edits a -200 region in the HBG1 or HBG2 promoter thereby disrupting a binding site for the LRF repressor.

6. The method of claim 5 wherein the gene editing platform introduces at least one mutation selected from the group consisting of -201C>T, -200C>T, -197C>T, -196C>T, -195C>T and -194C>T in the HBG1 or HBG2 promoter thereby disrupting a binding site for the LRF repressor.

7. The method of claim 1 wherein the gene editing edits a -115 region in the HBG1 or HBG2 promoter thereby disrupting a binding site for the BCL11A repressor.

8. The method of claim 7 wherein the gene editing platform introduces at least one mutation selected from the group consisting of -114C>T, -113C>T, -115C>T and -116C>T in the HBG1 or HBG2 promoter thereby disrupting a binding site for the BCL11A repressor.

9. The method of claim 1 wherein the eukaryotic cell is selected from the group consisting of hematopoietic progenitor cells, hematopoietic stem cells (HSCs), pluripotent cells and induced pluripotent stem cells (iPS)).

10. The method of claim 1 wherein the at least one base-editing enzyme comprises a nickase.

11. The method of claim 10 wherein the nickase comprises the amino acid sequence as set forth in SEQ ID NO: 3 or SEQ ID NO:33.

12. The method of claim 1 wherein the at least one base-editing enzyme is a cytidine deaminase or an adenosine deaminase.

13. The method of claim 12 wherein the cytidine deaminase or the adenosine deaminase comprises a variant of the amino acid sequence as set forth in SEQ ID NO:4-14.

14. The method of claim 1 wherein the at least one base-editing enzyme is ABEmax, AncBE4max, or evoCDA1-BE4max-NG.

15. The method of claim 1 wherein the at least one base-editing enzyme and the at least one guide RNA molecule is chosen according to Table B.

16. The method of claim 1 wherein a plurality of guide RNA molecules are designed for targeting a plurality of sequences in the HBG1 or HBG2 promoter.

17. The method of claim 1 wherein a plurality of base-editing enzymes and a plurality of guide RNA molecules are designed for targeting a plurality of sequences in the HBG1 or HBG2 promoter.

18. A method for increasing fetal hemoglobin levels in a subject in need thereof, comprising transplanting into the subject a therapeutically effective amount of a population of eukaryotic cells obtained by the method of claim 1.

19. The method of claim 18 wherein the subject has been diagnosed with a hemoglobinopathy.

20. The method of claim 9, wherein the pluripotent cells are embryonic stem (ES) cells.

21. The method of claim 10, wherein the nickase is a Cas9 nickase.

22. The method of claim 19 wherein the hemoglobinopathy is sickle cell disease or β-thalassemia.
